(19) United States
(12) Patent Application Publication
(10) Pub. No.: US 2025/0306386 A1
(43) Pub. Date: Oct. 2, 2025

(54) POSITIONING, STABILISING, AND INTERFACING STRUCTURES AND SYSTEM INCORPORATING SAME

(71) Applicant: ResMed Pty Ltd, Bella Vista (AU)

(72) Inventors: Ian Andrew LAW, Sydney (AU); Garth Alan BERRIMAN, Sydney (AU)

(21) Appl. No.: 19/088,863

(22) Filed: Mar. 24, 2025

(30) Foreign Application Priority Data
Mar. 26, 2024 (AU) 2024900806
Mar. 26, 2024 (AU) 2024900809

(51) Int. Cl. G02B 27/01

(52) U.S. Cl. CPC G02B 27/0176; G02B 2027/0154

(57) ABSTRACT

A forehead support for a head-mounted display system, comprising: a frame attachable to a head-mounted display unit of the head-mounted display system in use; and a resilient forehead pad attached to the frame and configured to engage the user's forehead in use, and wherein at least a portion of the frame is able to flex to conform to the shape of the user's forehead in use; and a shaping member attached to the frame, the shaping member configured to impart a shape to the frame.

### 1 CROSS-REFERENCE TO RELATED APPLICATIONS

N/A

### 2 BACKGROUND OF THE TECHNOLOGY

### 2.1 Field of the Technology

The present technology relates generally to head mounted displays, positioning and stabilizing structures, user interfacing structures, and other components for use in head mounted displays, associated head-mounted display assemblies and systems including a display unit and positioning and stabilizing structure, interfacing structures and or components, and methods. The present technology finds particular application in the use of immersive reality head mounted displays and is herein described in that context. It is to be appreciated that the present technology may have broader application and may be used in any type of head-mounted display arrangement including, but not limited to, virtual reality displays, augmented reality displays, and/or mixed reality displays.

### 2.2 Description of the Related Art

It is to be understood that, if any prior art is referred to herein, such reference does not constitute an admission that the prior art forms a part of the common general knowledge in the art, in Australia or any other country.

### 2.2.1 Immersive Technology

An immersive technology refers to technology that attempts to replicate or augment a physical environment through the means of a digital or virtual environment by creating a surrounding sensory feeling, thereby creating a sense of immersion.

In particular, an immersive technology provides the user visual immersion, and creates virtual objects and/or a virtual environment. The immersive technology may also provide immersion for at least one of the other five senses.

### 2.2.2 Virtual Reality

Virtual reality (VR) is a computer-generated three-dimensional image or environment that is presented to a user. In other words, the environment may be entirely virtual. Specifically, the user observes an electronic screen in order to observe virtual or computer generated images in a virtual environment. Since the created environment is entirely virtual, the user may be blocked and/or obstructed from interacting with their physical environment (e.g., they may be unable to hear and/or see the physical objects in the physical environment that they are currently located).

The electronic screen may be supported in the user's line of sight (e.g., mounted to the user's head). While observing the electronic screen, visual feedback output by the electronic screen and observed by the user may produce a virtual environment intended to simulate an actual environment. For example, the user may be able to look around (e.g., 360°) by pivoting their head or their entire body, and interact with virtual objects observable by the user through the electronic screen. This may provide the user with an immersive experience where the virtual environment provides stimuli to at least one of the user's five senses, and replaces the corresponding stimuli of the physical environment while the user uses the VR device. Typically, the stimuli relates at least to the user's sense of sight (i.e., because they are viewing an electronic screen), but other senses may also be included. The electronic screens are typically mounted to the user's head so that they may be positioned in close proximity to the user's eyes, which allows the user to easily observe the virtual environment.

The VR device may produce other forms of feedback in addition to, or aside from, visual feedback. For example, the VR device may include and/or be connected to a speaker in order to provide auditory feedback. The VR device may also include tactile feedback (e.g., in the form of haptic response), which may correspond to the visual and/or auditory feedback. This may create a more immersive virtual environment, because the user receives stimuli corresponding to more than one of the user's senses.

While using a VR device, a user may wish to limit to block ambient stimulation. For example, the user may want to avoid seeing and/or hearing the ambient environment in order to better process stimuli from the VR device in the virtual environment. Thus, VR devices may limit and/or prevent the user's eyes from receiving ambient light. In some examples, this may be done by providing a seal against the user's face. In some examples, a shield may be disposed proximate to (e.g., in contact or close contact with) the user's face, but may not seal against the user's face. In either example, ambient light may not reach the user's eyes, so that the only light observable by the user is from the electronic screen.

In other examples, the VR devices may limit and/or prevent the user's ears from hearing ambient noise. In some examples, this may be done by providing the user with headphones (e.g., noise cancelling headphones), which may output sounds from the VR device and/or limit the user from hearing noises from their physical environment. In some examples, the VR device may output sounds at a volume sufficient to limit the user from hearing ambient noise.

In any example, the user may not want to become overstimulated (e.g., by both their physical environment and the virtual environment). Therefore, blocking and/or limiting the ambient from stimulating the user assists the user in focusing on the virtual environment, without possible distractions from the ambient.

Different types of VR devices are described below. Generally, a single VR device may include at least two different classifications. For example, the VR device may be classified by its portability and by how the display unit is coupled to the rest of the interface. These classifications may be independent, so that classification in one group (e.g., the portability of the unit) does not predetermine classification into another group. There may also be additional categories to classify VR devices, which are not explicitly listed below.

### 2.2.2.1 Portability

### 2.2.2.1.1 Fixed Unit

In some forms, a VR device may be used in conjunction with a separate device, like a computer or video game console. This type of VR device may be fixed, since it cannot be used without the computer or video game console, and thus locations where it can be used are limited (e.g., by the location of the computer or video game console).

### 2.2.2.1.2 Portable Unit

In some forms, the VR device may be a self-contained unit, which includes a power source and sensors, so that the VR device does not need to be connected to a computer or video game console. This provides the user more freedom of use and movement. For example, the user is not limited to using the VR device near a computer or video game console, and could use the VR device outdoors, or in other environments that do not include computers or televisions.

### 2.2.2.2 Display Connection

### 2.2.2.2.1 Integrated Display Screen

In some forms, the display screen is an integral piece of the VR device, and generally cannot be detached or removed from the rest of the VR device.

The display screen may be fixed within a housing, and protected from damage. For example, the display screen may be completely covered by the housing, which may reduce the occurrence of scratches. Additionally, integrating display screen with the rest of the VR device eliminates the occurrence of losing the display screen.

In these forms, the display screen functions purely as an immersive technology display. The vast majority of “fixed units” will include an integrated display screen. “Portable units” may include an integrated display screen, or may include a removable display screen (described below).

### 2.2.2.2.2 Removable Display Screen

In some forms, the display screen is a separate structure that can be removed from the VR device, and used separately.

In some forms, a portable electronic device (e.g., a cell phone) may be selectively inserted into a housing of the VR device. The portable electronic device may include most or all of the sensors and/or processors, and may create a virtual environment through a downloadable app.

Portable electronic devices are generally light weight, and may not require the positioning and stabilizing structure to apply a large force to the user's head.

### 2.2.3 Augmented Reality

In some forms, augmented reality (AR) is a computer-generated three-dimensional image or environment that is presented to a user.

While similar to VR, AR differs in that the virtual environment created at least in part by the electronic screen is observed in combination with the user's physical environment. In other words, AR creates virtual objects in order to alter and/or enhance the user's physical environment with elements of a virtual environment. The result of AR is a combined environment that includes physical and virtual objects, and therefore an environment that is both physical and virtual.

For example, images created by the electronic screen may be overlayed into the user's physical environment. Only a portion of an AR combination environment presented to the user includes is virtual. Thus, the user may wish to continue to receive ambient stimulation from their physical environment while using an AR device (e.g., in order to continue to observe the physical or non-virtual component of the combination environment).

Since AR may be used with the user's physical environment, an AR device may not be electrically connected, or otherwise tethered, to a computer or video game console. Instead the AR device may include a battery, or other power source. This may provide the user with the greatest freedom of movement, so that they can explore a variety of physical environments while using the AR device.

This key difference between VR and AR may lead to different types of wearable electronic screens. As described above, a user of a VR device may wish to block ambient light, so the housing of the electronic screen may be opaque in order to limit or prevent ambient light from reaching the user. However, the user of an AR device may want to see the virtual environment blended with their actual environment. The electronic screen in an AR device may be similarly supported in front of the user's eyes, but, screens in AR devices may be transparent or translucent, and the screens may not be supported by an opaque housing (or opaque material may not substantially obstruct the user's line of sight). This may allow the user to continue receiving ambient stimulation, where the virtual environment is simultaneously present. Notwithstanding, some VR devices that do not have a transparent screen through which the user can see their real world surroundings may be configurable for AR by acquiring real-time video of the user's real-world surroundings from the user's perspective (e.g. with cameras on the display housing) and displaying it on the display screen.

Additionally, a person using an AR device may be more mobile than a person using a VR device (e.g., because an AR user can see their physical environment and/or are not tethered to a computer or video game console). Thus, a person using an AR device may wish to wear the device for an extended period of time, while also moving around (e.g., walking, running, biking, etc.). Including components, like batteries, on the AR device may make the AR device uncomfortable for the user's head and/or neck, and may discourage the user from wearing the AR device for long periods of time.

### 2.2.4 Mixed Reality

Mixed reality (MR) is similar to AR but may be more immersive because the MR device may provide the user more ways to interact with virtual objects or environment than an AR device. The virtual reality in MR may also be overlayed and/or blended with the user's physical environment. Unlike AR however, a user may be able to interact with the virtual environment akin to what occurs in VR. In other words, while AR may present only an computer generated image in the physical environment, MR may present the user with the same or similar computer generated image but allow for interaction with the image in the physical environment (e.g., using a hand to “grab” an object produced virtually). Thus, the virtual environment may further merge with a physical environment so that the combined environment better replicates an actual environment.

### 2.2.5 Head-Mounted Display Interface

A head-mounted display interface enables a user to have an immersive experience of a virtual environment and have broad application in fields such as communications, training, medical and surgical practice, engineering, and video gaming.

Different head-mounted display interfaces can each provide a different level of immersion. For example, some head-mounted display interfaces can provide the user with a total immersive experience. One example of a total immersive experience is virtual reality (VR). The head-mounted display interface can also provide partial immersion consistent with using an AR device.

VR head-mounted display interfaces typically are provided as a system that includes a display unit which is arranged to be held in an operational position in front of a user's face. The display unit typically includes a housing containing a display and a user interface structure constructed and arranged to be in opposing relation with the user's face. The user interface structure may extend about the display and define, in conjunction with the housing, a viewing opening to the display. The user interfacing structure may engage with the face and include a cushion for user comfort and/or be light sealing to block ambient light from the display. The head-mounted display system further comprises a positioning and stabilizing structure that is disposed on the user's head to maintain the display unit in position.

Other head-mounted display interfaces can provide a less than total immersive experience. In other words, the user can experience elements of their physical environment, as well as a virtual environment. Examples of a less than total immersive experience are augmented reality (AR) and mixed reality (MR).

AR and/or MR head-mounted display interfaces are also typically provided as a system that includes a display unit which is arranged to be held in an operational position in front of a user's face. Likewise, the display unit typically includes a housing containing a display and a user interface structure constructed and arranged to be in opposing relation with the user's face. The head-mounted display system of the AR and/or MR head-mounted display is also similar to VR in that it further comprises a positioning and stabilizing structure that is disposed on the user's head to maintain the display unit in position. However, AR and/or MR head-mounted displays do not include a cushion that totally seals ambient light from the display, since these less than total immersive experience require an element of the physical environment. Instead, head-mounted displays in augmented and/or mixed allow the user to see the physical environment in combination with the virtual environment.

In any types of immersive technology, it is important that the head-mounted display interface is comfortable in order to allow the user to wear the head-mounted display for extended periods of time. Additionally, it is important that the display is able to provide changing images with changing position and/or orientation of the user's head in order to create an environment, whether partially or entirely virtual, that is similar to or replicates one that is entirely physical.

### 2.2.5.1 Interfacing Structure

The head-mounted displays may include a user interfacing structure. Since it is in direct contact with the user's face, the shape and configuration of the interfacing portion can have a direct impact on the effectiveness and comfort of the display unit.

The design of a user interfacing structure presents a number of challenges. The face has a complex three-dimensional shape. The size and shape of noses and heads varies considerably between individuals. Since the head includes bone, cartilage and soft tissue, different regions of the face respond differently to mechanical forces.

One type of interfacing structure extends around the periphery of the display unit and is intended to seal against the user's face when force is applied to the user interface with the interfacing structure in confronting engagement with the user's face. The interfacing structure may include a pad made of a polyurethane (PU). With this type of interfacing structure, there may be gaps between the interfacing structure and the face, and additional force may be required to force the display unit against the face in order to achieve the desired contact.

The regions not engaged at all by the user interface may allow gaps to form between the facial interface and the user's face through which undesirable light pollution may ingress into the display unit (e.g., particularly when using virtual reality). The light pollution or “light leak” may decrease the efficacy and enjoyment of the overall immersive experience for the user. In addition, previous systems may be difficult to adjust to enable application for a wide variety of head sizes. Further still, the display unit and associated stabilizing structure may often be relatively heavy and may be difficult to clean which may thus further limit the comfort and useability of the system.

Another type of interfacing structure incorporates a flap seal of thin material positioned about a portion of the periphery of the display unit so as to provide a sealing action against the face of the user. Like the previous style of interfacing structure, if the match between the face and the interfacing structure is not good, additional force may be required to achieve a seal, or light may leak into the display unit in-use. Furthermore, if the shape of the interfacing structure does not match that of the user, it may crease or buckle in-use, giving rise to undesirable light penetration.

A user interface may be partly characterised according to the design intent of where the interfacing structure is to engage with the face in-use. Some interfacing structures may be limited to engaging with regions of the user's face that protrude beyond the arc of curvature of the face engaging surface of the interfacing structure. These regions may typically include the user's forehead and cheek bones. This may result in user discomfort at localised stress points. Other facial regions may not be engaged at all by the interfacing structure or may only be engaged in a negligible manner that may thus be insufficient to increase the translation distance of the clamping pressure. These regions may typically include the sides of the user's face, or the region adjacent and surrounding the users nose. To the extent to which there is a mismatch between the shape of the users' face and the interfacing structure, it is advantageous for the interfacing structure or a related component to be adaptable in order for an appropriate contact or other relationship to form.

### 2.2.5.2 Positioning and Stabilizing

To hold the display unit in its correct operational position, the head-mounted display system further comprises a positioning and stabilizing structure that is disposed on the user's head. These structures may be responsible for providing forces to counter gravitational forces of the head-mounted display and/or interfacing structure. In the past these structures have been formed from expandable rigid structures that are typically applied to the head under tension to maintain the display unit in its operational position. Such systems have been prone to exert a clamping pressure on the user's face which can result in user discomfort at localised stress points. Also, previous systems may be difficult to adjust to allow wide application head sizes. Further, the display unit and associated stabilizing structure are often heavy, difficult to clean which further limit the comfort and useability of the system.

Certain other head mounted display systems may be functionally unsuitable for the present field. For example, positioning and stabilizing structures designed for ornamental and visual aesthetics may not have the structural capabilities to maintain a suitable pressure around the face. For example, an excess of clamping pressure may cause discomfort to the user, or alternatively, insufficient clamping pressure on the users' face may not effectively seal the display from ambient light.

Certain other head mounted display systems may be uncomfortable or impractical for the present technology. For example, if the system is used for prolonged time periods.

As a consequence of these challenges, some head mounted displays suffer from being one or more of obtrusive, aesthetically undesirable, costly, poorly fitting, difficult to use, and uncomfortable especially when worn for long periods of time or when a user is unfamiliar with a system. Wrongly sized positioning and stabilizing structures can give rise reduced comfort and in turn, shortened periods of use.

Therefore, an interfacing portion of a user interface used for the fully immersive experience of a virtual environment are subject to forces corresponding to the movement of a user during the experience.

### 2.2.5.3 Materials

Materials used in head mounted display assemblies have included dense foams for contacting portions in the interfacing structures, rigid shells for the housings, and positioning and stabilizing structures formed from rigid plastic clamping structures. These materials have various drawbacks including not permitting the skin covered by the material to breath, being inflexible, difficult to clean and to prone trapping bacteria. As a result, products made with such material may be uncomfortable to wear for extended periods of time, causes skin irritation in some individuals and limit the application of the products.

### 3 BRIEF SUMMARY OF THE TECHNOLOGY

The present technology may be directed toward providing positioning and stabilizing structures used in the supporting, stabilizing, mounting, utilizing, and/or securing of a head-mounted display having one or more of improved comfort, cost, efficacy, ease of use and manufacturability.

A first aspect of the present technology relates to apparatuses used in the supporting, stabilizing, mounting, utilizing, and/or securing of a head-mounted display.

Another aspect of the present technology relates to methods used in the supporting, stabilizing, mounting, utilizing, and/or securing of a head-mounted display.

Another aspect is a positioning and stabilizing structure for a head-mounted display that comprising a rear (or posterior) support structure (or portion) arranged, in use, to contact a posterior region of the user's head.

In some forms, the posterior support portion or at least a portion thereof is disposed posterior of the otobasion superior of the user.

In some forms, the posterior support portion is biased into contact with the occipital region of the user.

In some forms, the positioning and stabilizing structure further comprises opposing connectors that are disposed on opposing sides of, and extending along the temporal regions of, the user's head to interconnect the posterior support portion to the head-mounted display unit. In some forms the positioning and stabilising structure comprises an anterior support portion connecting the posterior support portion to the head-mounted display unit.

The present technology may also be directed toward providing interfacing structures used in the supporting, cushioning, stabilizing, positioning, and/or sealing a head-mounted display in opposing relation with the user's face.

Another aspect relates to apparatuses used in the supporting, cushioning, stabilizing, positioning, and/or sealing a head-mounted display in opposing relation with the user's face.

Another aspect relates to methods used in supporting, cushioning, stabilizing, positioning, and/or sealing a head-mounted display in opposing relation with the user's face.

### 3.1 Flexible Frames

Another aspect of the present technology relates to a head-mounted display system, comprising:
a head-mounted display unit comprising a display unit housing comprising a display; and
a positioning and stabilising structure constructed and arranged to hold the head-mounted display unit in an operable position on the user's head in use;
wherein the positioning and stabilising structure comprises a forehead support, the forehead support comprising a frame attached to the head-mounted display unit in use and a resilient forehead pad attached to the frame and configured to engage the user's forehead in use, and wherein at least a portion of the frame is able to flex to conform to the shape of the user's forehead in use;
wherein the forehead support comprises a shaping member attached to the frame, the shaping member configured to impart a shape to the frame.

Another aspect of the present technology relates to a forehead support for a head-mounted display system, comprising:
a frame attachable to a head-mounted display unit of the head-mounted display system in use; and
a resilient forehead pad attached to the frame and configured to engage the user's forehead in use, and wherein at least a portion of the frame is able to flex to conform to the shape of the user's forehead in use; and
a shaping member attached to the frame, the shaping member configured to impart a shape to the frame.

In examples:
the shaping member is more rigid than the frame;
the shaping member is substantially rigid;
the shaping member is configured to attach to an anterior surface of the frame;
the shaping member comprises a plate;
the shaping member comprises a skeleton;
the shape imparted to the frame by the shaping member corresponds to the shape of user's forehead;
the shape imparted to the frame by the shaping member matches the shape of the user's forehead;
the shaping member is formed based on a scan of the user's forehead; and/or
the shaping member is formed from 3D printing.

In further examples:
the frame when shaped by the shaping member is configured to bias the forehead pad into a shape that is more curved than the user's forehead such that in use the forehead pad is conformed to the shape of the user's head;
the frame comprises a central portion structured to attach to the display unit housing;
the frame comprises a pair of lateral portions having lateral ends able to move with respect to the central portion, the shaping member constructed and arranged to conform the lateral portions to the shape of the user's forehead in use;
the lateral ends are each able to be moved by the shaping member at least partially medially and laterally;
the lateral ends are each able to be moved by the shaping member at least partially anteriorly and posteriorly;
each of the lateral portions comprises a superior lateral portion and an inferior lateral portion and the lateral ends comprise a superior lateral end of each superior lateral portion and an inferior lateral end of each inferior lateral portion, each superior lateral portion being able to be flexed by the shaping member independently of the respective inferior lateral portion;
the frame comprises a superior medial portion having a superior end able to move with respect to the central portion of the frame, the shaping member constructed and arranged to conform the superior medial portion to the shape of the user's forehead in use;
the superior end is able to be moved by the shaping member at least partially anteriorly and posteriorly;
the frame comprises an inferior medial portion having an inferior end able to move with respect to the central portion of the frame, the shaping member constructed and arranged to conform the inferior medial portion to the shape of the user's forehead in use;
the inferior end is able to be moved by the shaping member at least partially anteriorly and posteriorly;
the central portion comprises a connection portion positioned anteriorly with respect to the lateral portions, the connection portion configured to be attached to the display unit housing of the head-mounted display unit;
the forehead pad comprises a foam layer;
the forehead pad comprises a textile user-contacting layer;
the textile user-contacting layer is attached to a silicone backing layer;
the textile user-contacting layer is attached to and supported by a silicone peripheral portion connected to the frame; and/or
the forehead pad comprises a silicone user-contacting layer.

Another aspect of the present technology relates to a head-mounted display system, comprising:
a head-mounted display unit comprising a display unit housing comprising a display; and
a positioning and stabilising structure constructed and arranged to hold the head-mounted display unit in an operable position on the user's head in use;
wherein the positioning and stabilising structure comprises a posterior support portion configured to engage a posterior portion of the user's head in use, and a pair of lateral support portions connecting between the posterior support portion and the head-mounted display unit in use;
wherein the posterior support portion of the positioning and stabilising structure comprises a posterior support pad assembly comprising a posterior frame connected to the lateral support portions, and a posterior pad being connected to the posterior frame, the posterior pad being configured to contact the user's head in use;
wherein the posterior support pad assembly comprises a shaping member attached to the posterior frame, the shaping member configured to impart a shape to the posterior frame.

Another aspect of the present technology relates to a posterior support pad assembly for a head-mounted display system, comprising:
a posterior frame connectable to a pair of lateral support portions of the head-mounted display system for connecting the posterior support pad assembly to a head-mounted display unit of the head-mounted display system,
a posterior pad being connected to the posterior frame, the posterior pad being configured to contact the user's head in use; and
a shaping member attached to the posterior frame, the shaping member configured to impart a shape to the posterior frame.

In examples:
the shaping member is more rigid than the posterior frame;
the shaping member is substantially rigid;
the shaping member is configured to attach to a posterior surface of the posterior frame;
the shaping member comprises a plate;
the shaping member comprises a skeleton;
the shape imparted to the posterior frame by the shaping member corresponds to the shape of a posterior surface of the user's head;
the shape imparted to the posterior frame by the shaping member matches the shape of the posterior surface of the user's head;
the shaping member is formed based on a scan of the user's head;
the shaping member is formed from 3D printing;
the posterior frame when shaped by the shaping member is configured to bias the posterior pad into a shape that is more curved than the shape of the posterior surface of the user's head such that in use the posterior pad is conformed to the posterior surface of the user's head;
the posterior frame comprises a central portion connected to the lateral support portions;
the posterior frame comprises a pair of lateral portions having lateral ends able to move with respect to the central portion, the shaping member being constructed and arranged to conform the lateral portions to the shape of the posterior surface of the user's head in use;
the lateral ends are each able to be moved by the shaping member at least partially medially and laterally;
the lateral ends are each able to be moved by the shaping member at least partially anteriorly and posteriorly;
the posterior frame comprises a superior medial portion having a superior end able to move with respect to the central portion of the posterior frame, the shaping member being constructed and arranged to conform the superior medial portion to the shape of posterior surface of the user's head in use;
the superior end is able to be moved by the shaping member at least partially anteriorly and posteriorly;
the posterior frame comprises an inferior medial portion having an inferior end able to move with respect to the central portion of the posterior frame, the shaping member being constructed and arranged to conform the inferior medial portion to the shape of the posterior surface of the user's head in use;
the inferior end is able to be moved by the shaping member at least partially anteriorly and posteriorly;
the posterior support pad comprises a foam layer;
the posterior support pad comprises a textile user-contacting layer;
the textile user-contacting layer is attached to a silicone backing layer;
the textile user-contacting layer is attached to and supported by a silicone peripheral portion connected to the posterior frame; and/or
the posterior support pad comprises a silicone user-contacting layer.

### 3.2 Headgear Tension

Another aspect of the present technology relates to a head-mounted display system, comprising:
a head-mounted display unit comprising:
a display unit housing comprising a display; and
an interfacing structure connected to the display unit housing and constructed and arranged to engage the user's face in use; and
a positioning and stabilising structure constructed and arranged to hold the head-mounted display unit in an operable position on the user's head in use, the positioning and stabilising structure comprising:
   a band strap portion constructed and arranged to encircle the user's head in use to engage a region of the user's head overlying the frontal bone and engage a posterior region of the head;
   wherein the head-mounted display unit is attached to the band strap portion at a pair of lateral connections and the band strap portion is configured to be in tension in use;
   wherein the head-mounted display unit is able to be lifted upwardly by the user away from a viewing position to remove the head-mounted display system from the user's head, and wherein when the head-mounted display unit is lifted upwardly away from the viewing position the head-mounted display unit rotates at the lateral connections which relieves tension in the band strap portion.

In examples:
the band strap portion forms an occipital strap portion constructed and arranged to engage the posterior region of the user's head, the posterior region of the user's head overlying or lying inferior to the occipital bone;
the band strap portion is formed from an anterior portion of the band strap portion and a posterior portion of the band strap portion, the anterior portion and posterior portion of the band strap portion each having ends at the lateral connections, wherein at each lateral connection the head-mounted display unit connects to respective ends of the anterior portion and the posterior portion of the band strap portion;
wherein when the head-mounted display unit is in the viewing position, at each lateral connection the respective ends of the anterior portion and posterior portion of the band strap portion are pulled towards each other, and when the head-mounted display unit is lifted upwardly away from the viewing position, at each lateral connection the respective ends of the anterior portion and posterior portion of the band strap portion move apart;
the head-mounted display unit may comprise a pair of arms each connected to the band strap portion at a respective lateral connection;
each arm is pivotably attached to one or both of the anterior portion and posterior portion of the band strap portion;
each arm connects to the anterior portion and posterior portion of the band strap portion at respective connection points between said arm and the anterior portion and posterior portion of the band strap portion;
each connection point is a rotatable connection;
the connection points on the anterior portion of the band strap portion are positioned proximate an inferior edge of the anterior portion of the band strap portion; and/or
the connection points on the posterior portion of the band strap portion are positioned proximate a superior edge of the posterior portion of the band strap portion;
the positioning and stabilising structure comprises at least one cam mechanism that tensions the band strap portion when the head-mounted display unit is moved into the viewing position and releases tension in the band strap portion when the head-mounted display unit is lifted upwardly away from the viewing position;
the cam mechanism comprises a cam and one of the pair of arms rotates the cam during movement of the head-mounted display unit towards or away from the viewing position to operate the cam mechanism;
the cam mechanism is constructed and arranged to resist movement of the head-mounted display unit away from the viewing position when the head-mounted display unit is in the viewing position;
the cam mechanism resists movement of the head-mounted display unit away from the viewing position by generating an initial increase in tension in the band strap portion when the user lifts the head-mounted display unit upwardly away from the viewing position, wherein after the initial increase in tension the cam mechanism relieves tension in the band strap portion;
the cam mechanism operates to impart or allow relative movement of the respective ends of the anterior portion and posterior portion of the band strap portion;
the cam mechanism comprises a cam and a tether connecting one of the anterior portion of the band strap portion and the posterior portion of the band strap portion to the cam, wherein rotation of the cam during movement of the head-mounted display unit towards the viewing position pulls said respective one of the anterior portion or posterior portion towards the cam to increase tension in the band strap portion; and
the cam mechanism comprises a cam and a tether connecting the anterior portion of the band strap portion to the posterior portion of the band strap portion, the cam constructed and arranged to engage the tether to draw the anterior portion and posterior portion of the band strap portion together during movement of the head-mounted display unit towards the viewing position;
the tether is constructed from an elastically extendable material.

Another form of the present technology comprises a head mounted display system for a person comprising:
a head-mounted display unit comprising a display;
a control system for operation of the head-mounted display system; and
a positioning and stabilizing structure configured to configured to hold the head-mounted display unit anterior to a user's eyes such that the display is viewable by the user in use.

The head-mounted display system may be helmet mounted, may be configured for virtual reality display, may be configured for augmented reality display, may be configured for mixed reality display.

Another form of the present technology comprises a head mounted display interface comprising:
an electronic display screen configured to output multiple images to a user;
a display housing configured to at least partially house the electronic display screen; and
a positioning and stabilizing structure coupled to the display housing and supporting the display housing and the electronic display screen in an operating position, the positioning and stabilizing structure being configured to provide a force against a user's head in order to counteract a moment produced by a combined weight of the electronic display screen and the display housing, and maintain a position of the electronic display screen anterior to the user's eyes while in the operating position;
wherein the positioning and stabilising structure is substantially as described in any example disclosed herein.

Another form of the present technology comprises a positioning and stabilizing structure for supporting an electronic display screen of a head-mounted display interface, the positioning and stabilizing structure being configured to provide a force against a user's head in order to counteract a moment produced by a weight of the electronic display screen, and maintain a position of the electronic display screen anterior to the user's eyes while in use, the positioning and stabilizing structure comprising:
a rear strap configured to contact a region of the user's head posterior to the coronal plane of the user's head, the rear strap configured to anchor the head-mounted display interface to the user's head.

Another aspect of one form of the present technology is a positioning and stabilizing structure that is constructed with a shape which is complementary to that of an intended wearer.

Another aspect of one form of the present technology is an interfacing structure that is constructed with a shape which is complementary to that of an intended wearer.

The methods, systems, devices and apparatus described may be implemented so as to improve the functionality of a head-mounted display, such as an electronic display or computer. Moreover, the described methods, systems, devices and apparatus can provide improvements in the technological field of virtual reality, augmented reality, and/or mixed reality.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the figures of the accompanying drawings, in which like reference numerals refer to similar elements including:

### 4.1 Head-Mounted Display Systems

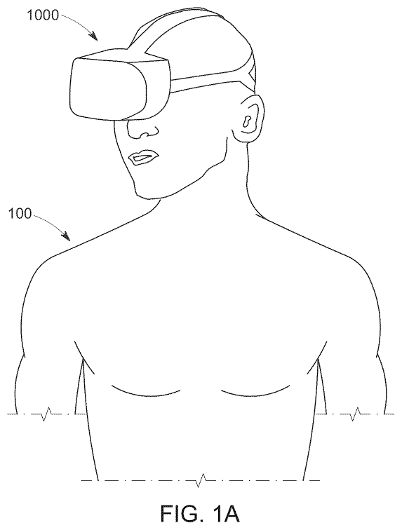

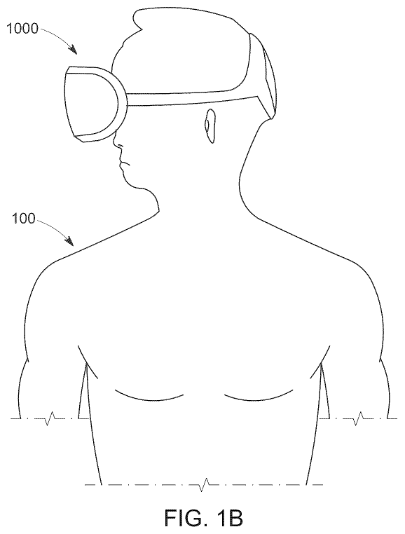

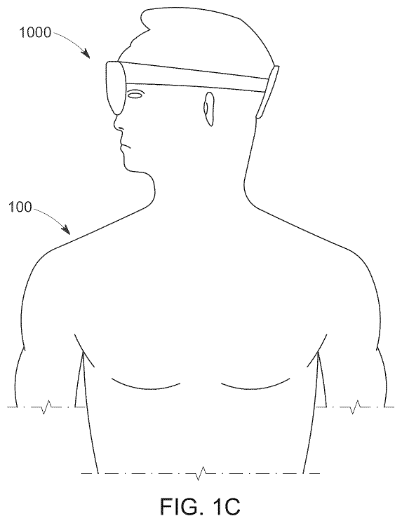

FIG. 1A shows a system including a user 100 wearing a head-mounted display system 1000, in the form of a face-mounted, virtual reality (VR) headset, displaying various images to the user 100. The user is standing while wearing the head-mounted display system 1000.

FIG. 1B shows a system including a user 100 wearing a head-mounted display system 1000, in the form of a floating virtual reality (VR) headset, displaying various images to the user. The user is sitting while wearing the display interface 100.

FIG. 1C shows a system including a user 100 wearing a head-mounted display system 1000, in the form of a floating augmented reality (AR) headset, displaying various images to the user. The user is standing while wearing the head-mounted display system 1000.

### 4.2 Display System and Facial Anatomy

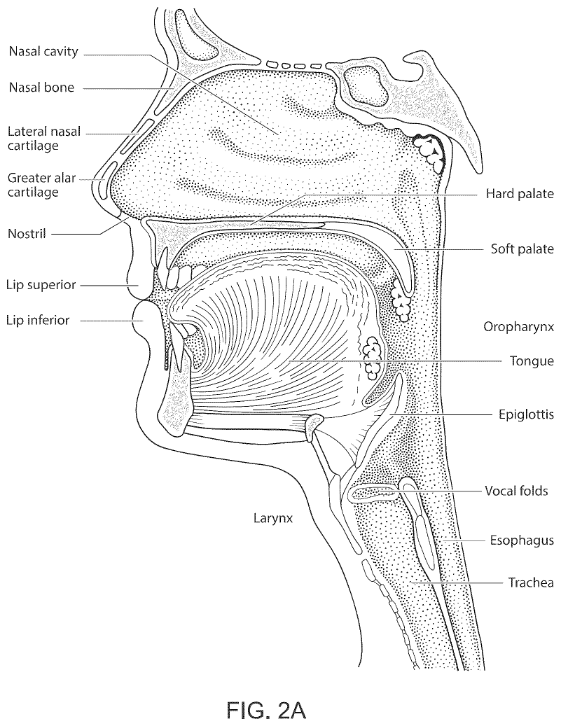

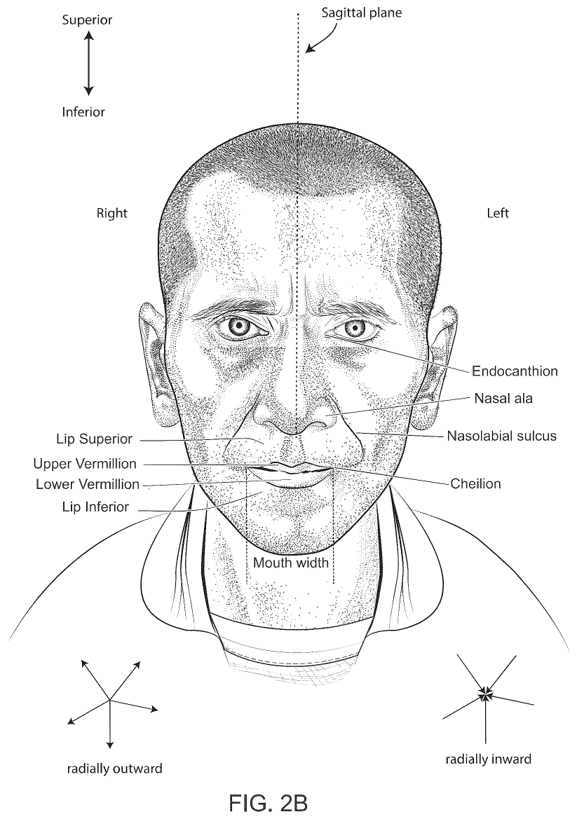

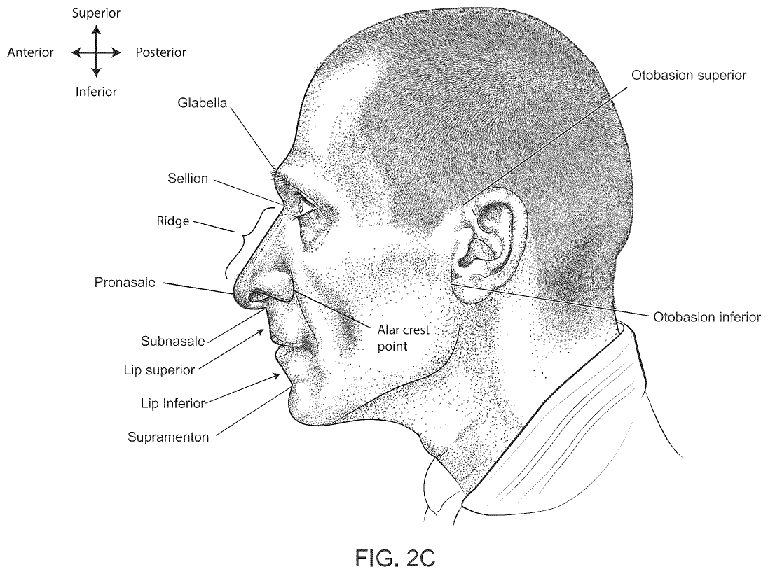

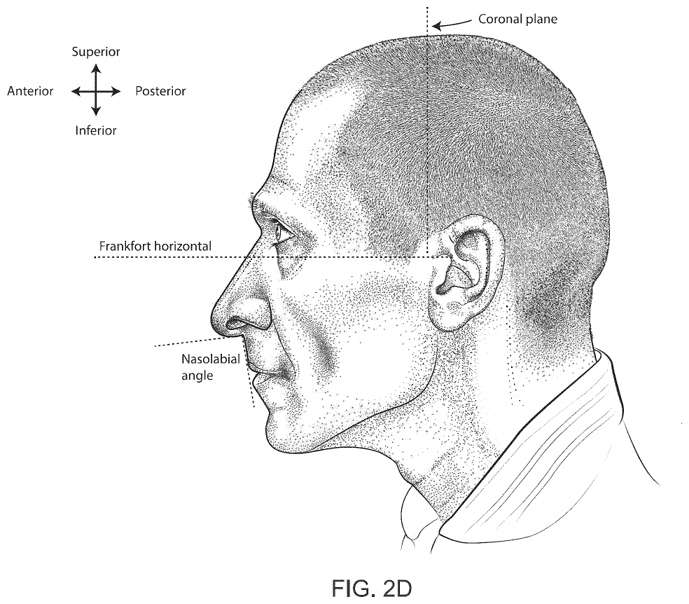

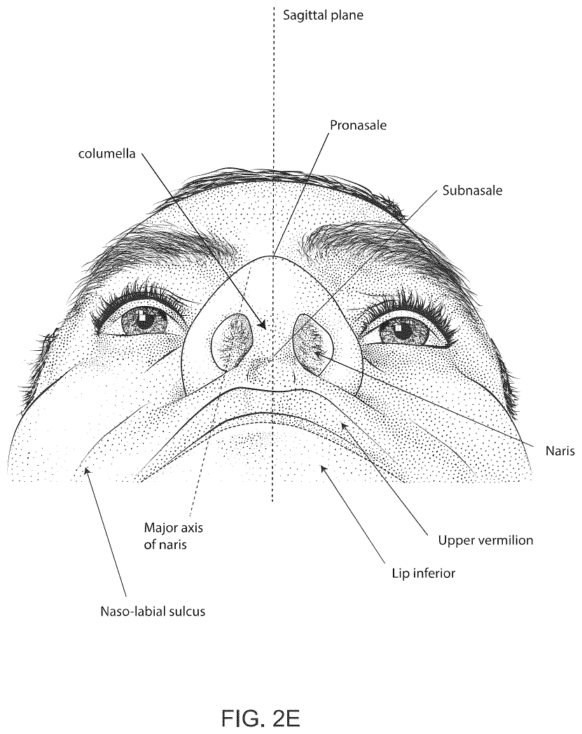

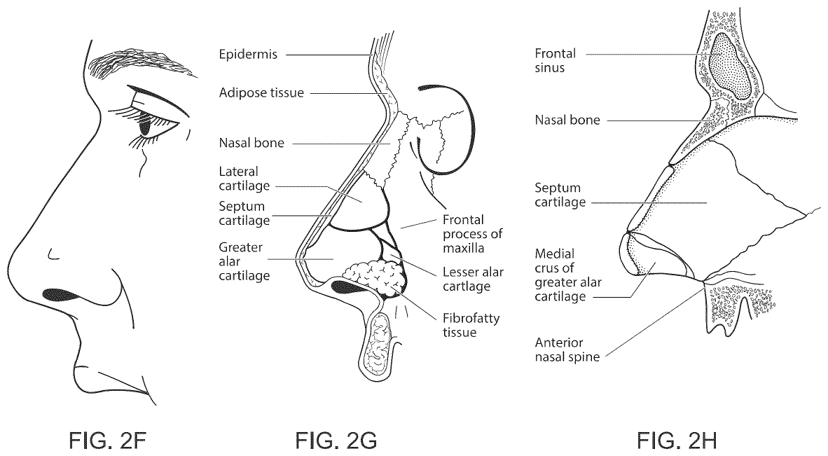

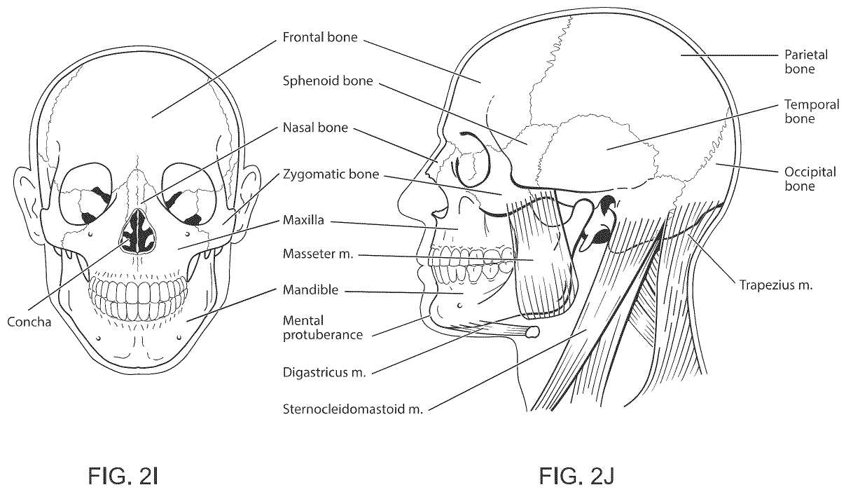

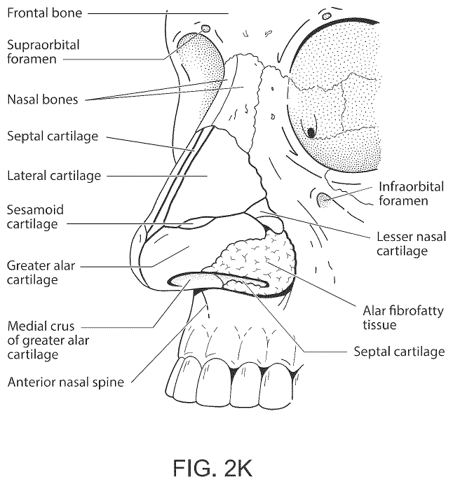

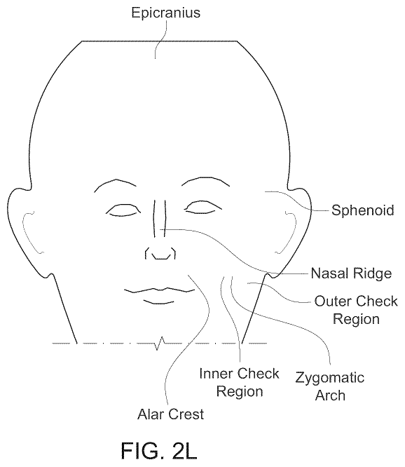

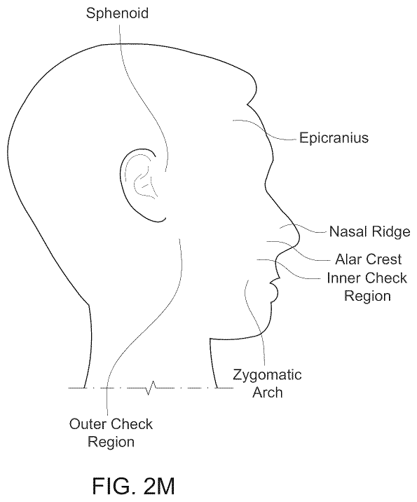

FIG. 2A shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

FIG. 2B is a front view of a face with several features of surface anatomy identified including the lip superior, upper vermilion, lower vermilion, lip inferior, mouth width, endocanthion, a nasal ala, nasolabial sulcus and cheilion. Also indicated are the directions superior, inferior, radially inward and radially outward.

FIG. 2C is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2D is a further side view of a head. The approximate locations of the Frankfort horizontal and nasolabial angle are indicated. The coronal plane is also indicated.

FIG. 2E shows a base view of a nose with several features identified including naso-labial sulcus, lip inferior, upper Vermilion, naris, subnasale, columella, pronasale, the major axis of a naris and the midsagittal plane.

FIG. 2F shows a side view of the superficial features of a nose.

FIG. 2G shows subcutaneal structures of the nose, including lateral cartilage, septum cartilage, greater alar cartilage, lesser alar cartilage, sesamoid cartilage, nasal bone, epidermis, adipose tissue, frontal process of the maxilla and fibrofatty tissue.

FIG. 2H shows a medial dissection of a nose, approximately several millimeters from the midsagittal plane, amongst other things showing the septum cartilage and medial crus of greater alar cartilage.

FIG. 2I shows a front view of the bones of a skull including the frontal, nasal and zygomatic bones. Nasal concha are indicated, as are the maxilla, and mandible.

FIG. 2J shows a lateral view of a skull with the outline of the surface of a head, as well as several muscles. The following bones are shown: frontal, sphenoid, nasal, zygomatic, maxilla, mandible, parietal, temporal and occipital. The mental protuberance is indicated. The following muscles are shown: digastricus, masseter, sternocleidomastoideo trapezius.

FIG. 2K shows an anterolateral view of a nose. The following bones are shown: frontal, supraorbital foramen, nasal, septal cartilage, lateral cartilage, orbit and infraorbital foramen.

FIG. 2L shows another front view of the face with several features of surface anatomy identified including the epicranius, the sphenoid, the nasal ridge, the outer and inner cheek regions, the zygomatic arch, and the alar crest.

FIG. 2M shows another side view of the face with several features of surface anatomy identified including the epicranius, the sphenoid, the nasal ridge, the outer and inner cheek regions, the zygomatic arch, and the alar crest.

### 4.3 Shape of Structures

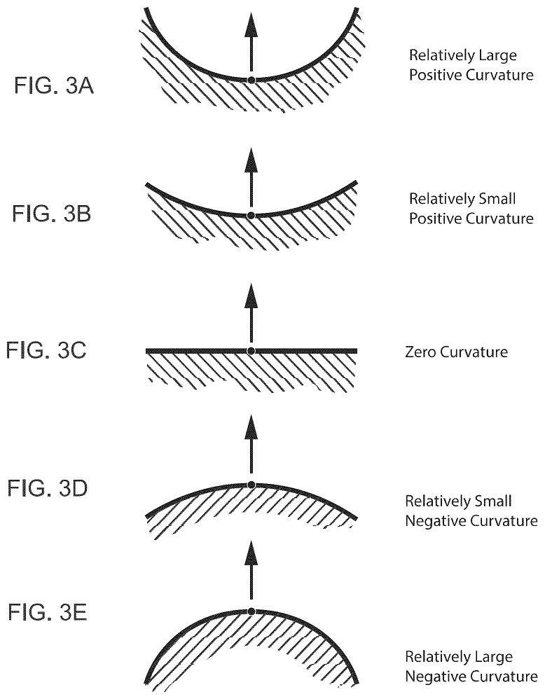

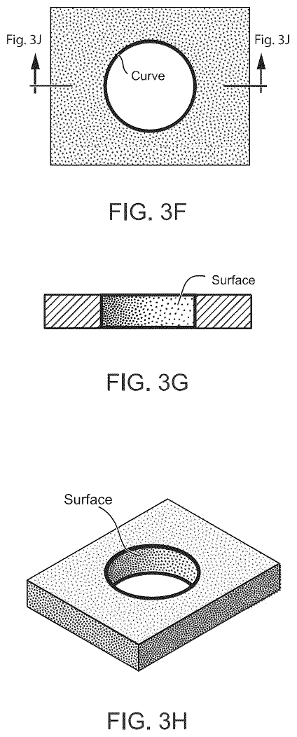

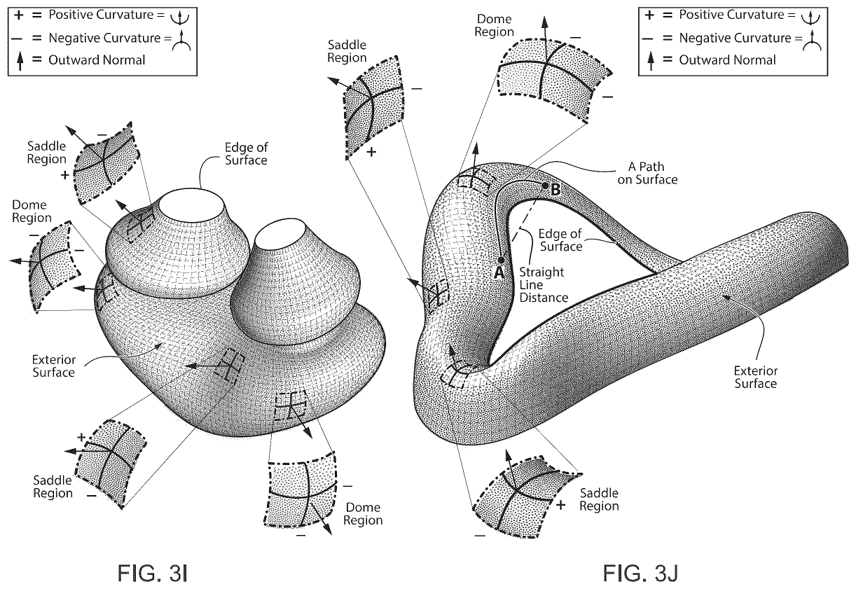

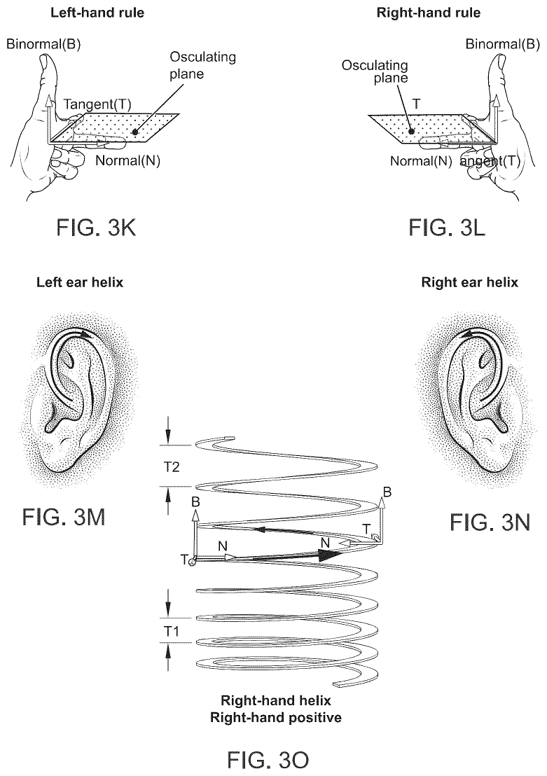

FIG. 3A shows a schematic of a cross-section through a structure at a point. An outward normal at the point is indicated. The curvature at the point has a positive sign, and a relatively large magnitude when compared to the magnitude of the curvature shown in FIG. 3B.

FIG. 3B shows a schematic of a cross-section through a structure at a point. An outward normal at the point is indicated. The curvature at the point has a positive sign, and a relatively small magnitude when compared to the magnitude of the curvature shown in FIG. 3A.

FIG. 3C shows a schematic of a cross-section through a structure at a point. An outward normal at the point is indicated. The curvature at the point has a value of zero.

FIG. 3D shows a schematic of a cross-section through a structure at a point. An outward normal at the point is indicated. The curvature at the point has a negative sign, and a relatively small magnitude when compared to the magnitude of the curvature shown in FIG. 3E.

FIG. 3E shows a schematic of a cross-section through a structure at a point. An outward normal at the point is indicated. The curvature at the point has a negative sign, and a relatively large magnitude when compared to the magnitude of the curvature shown in FIG. 3D.

FIG. 3F shows the surface of a structure, with a one dimensional hole in the surface. The illustrated plane curve forms the boundary of a one dimensional hole.

FIG. 3G shows a cross-section through the structure of FIG. 3F. The illustrated surface bounds a two dimensional hole in the structure of FIG. 3F.

FIG. 3H shows a perspective view of the structure of FIG. 3F, including the two dimensional hole and the one dimensional hole. Also shown is the surface that bounds a two dimensional hole in the structure of FIG. 3F.

FIGS. 31I-3J shows a seal forming structure. An exterior surface of the cushion is indicated. An edge of the surface is indicated. A path on the surface between points A and B is indicated. A straight-line distance between A and B is indicated. Two saddle regions and a dome region are indicated.

FIG. 3K illustrates a left-hand rule.

FIG. 3L illustrates a right-hand rule.

FIG. 3M shows a left ear, including the left ear helix.

FIG. 3N shows a right ear, including the right ear helix.

FIG. 3O shows a right-hand helix.

### 4.4 Head-Mounted Virtual Reality Display

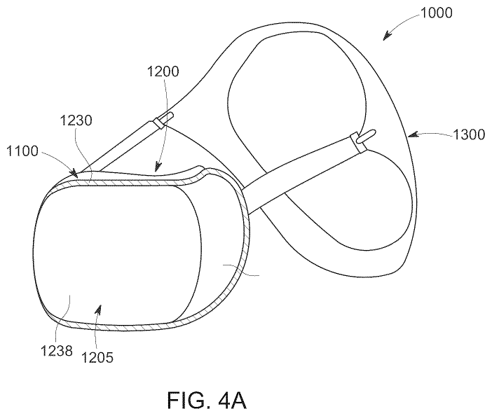

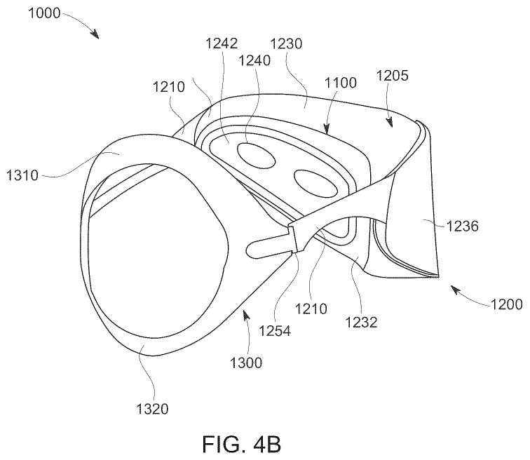

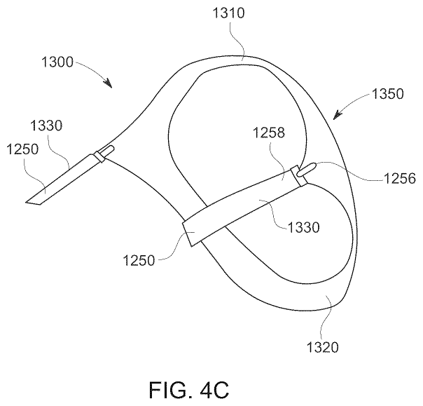

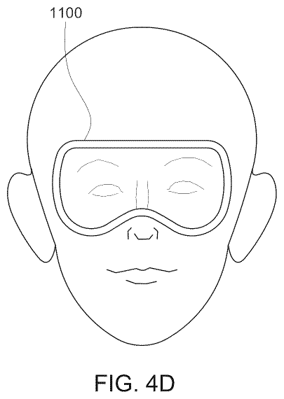

FIG. 4A shows a front perspective view of a head-mounted display interface in accordance with one form of the present technology.

FIG. 4B shows a rear perspective view of the head-mounted display of FIG. 4A.

FIG. 4C shows a perspective view of a positioning and stabilizing structure used with the head-mounted display of FIG. 4A.

FIG. 4D shows a front view of a user's face, illustrating a location of an interfacing structure, in use.

### 4.5 Head-Mounted Augmented Reality Display

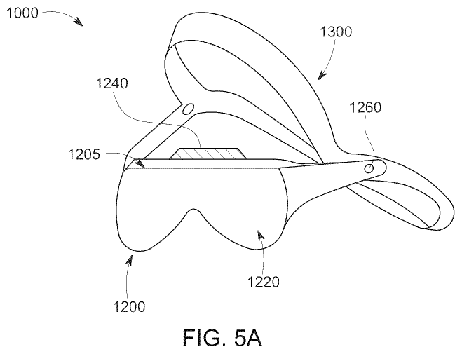

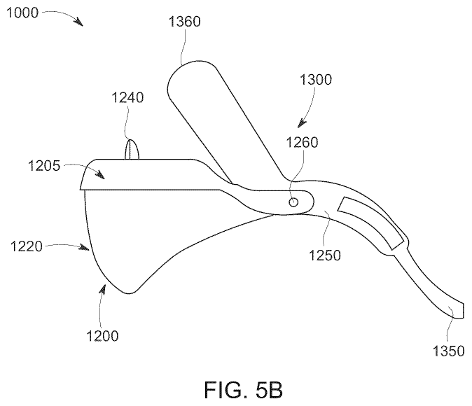

FIG. 5A shows a front perspective view of a head-mounted display interface in accordance with one form of the present technology.

FIG. 5B shows a side view of the head-mounted display interface of FIG. 5A.

### 4.6 Controls

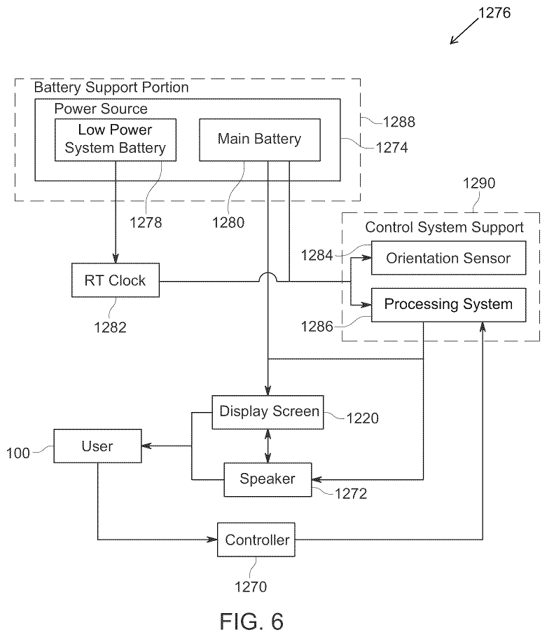

FIG. 6 shows a schematic view of a control system of one form of the present technology.

### 4.7 Head-Mounted Display System

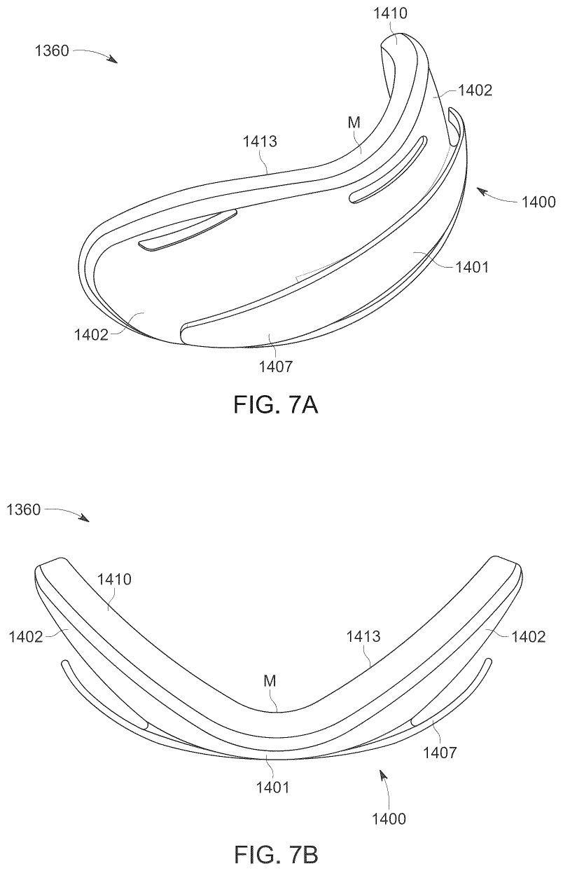

FIG. 7A is a perspective view of a forehead support according to one example of the present technology.

FIG. 7B is a top view of the forehead support shown in FIG. 7A.

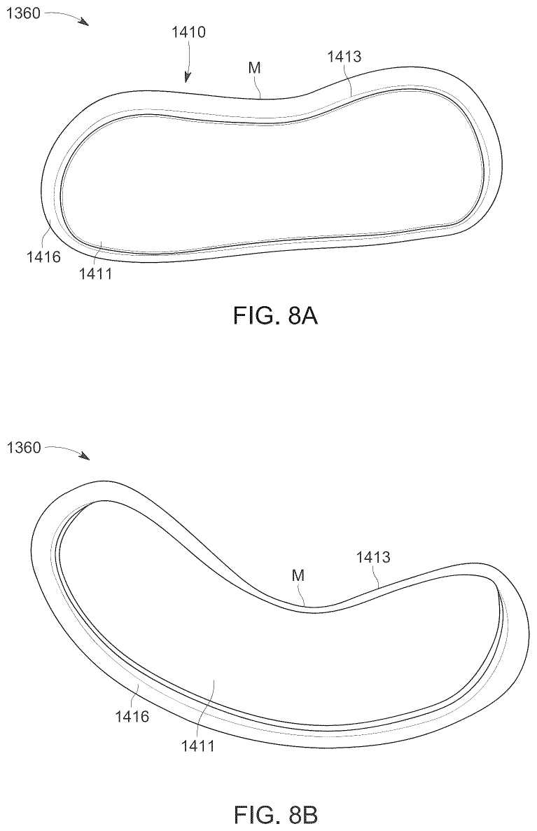

FIG. 8A is a posterior view of the forehead support shown in FIG. 7A.

FIG. 8B is an inferior posterior perspective view of the forehead support shown in FIG. 7A.

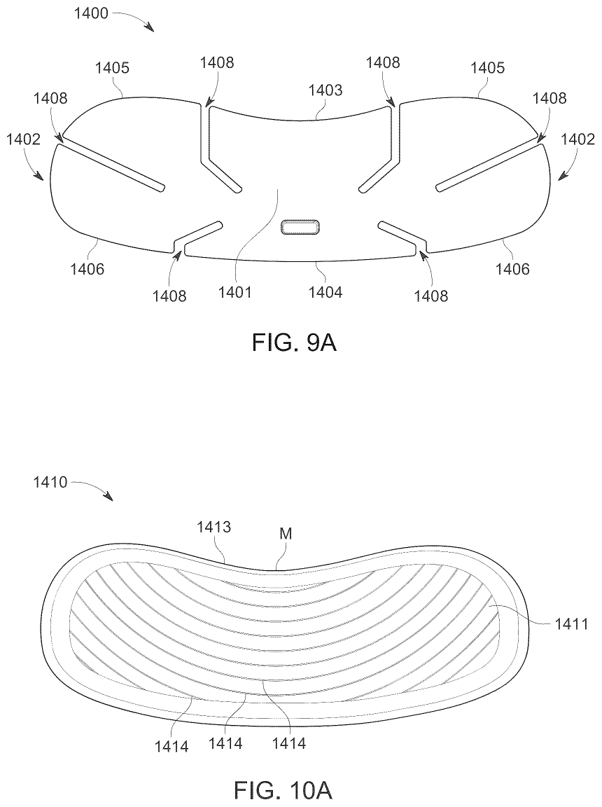

FIG. 9A is a posterior view of a frame for a forehead support according to another example of the present technology.

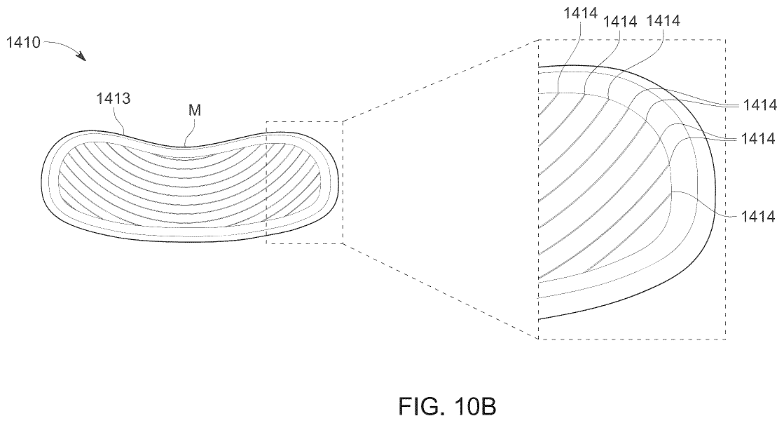

FIG. 10A is a posterior view of a forehead pad according to another example of the present technology.

FIG. 10B is a detail view of a surface of the forehead pad shown in FIG. 10A.

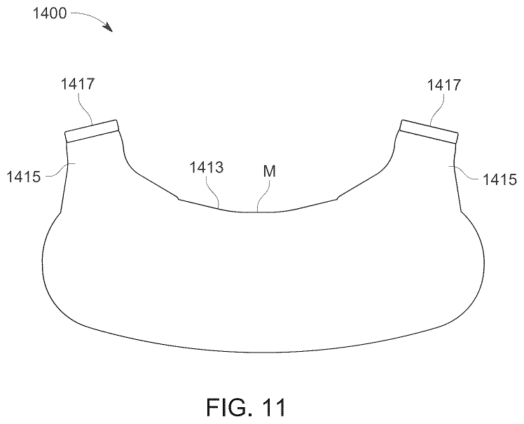

FIG. 11 is a schematic view of a frame of a forehead support according to another example of the present technology.

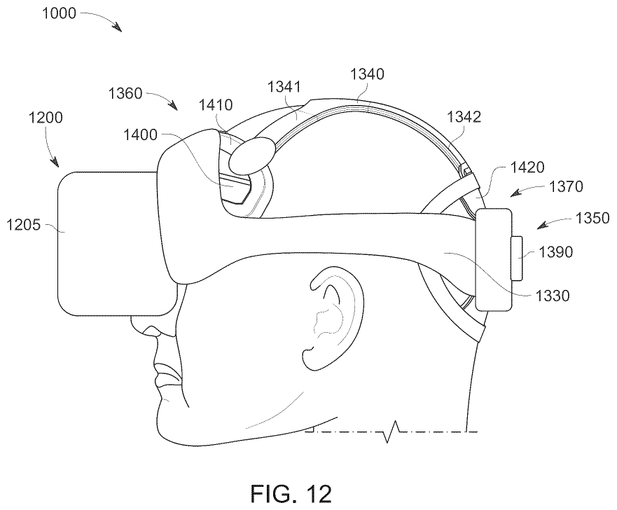

FIG. 12 is a lateral view of a head-mounted display system according to an example of the present technology, worn by a user.

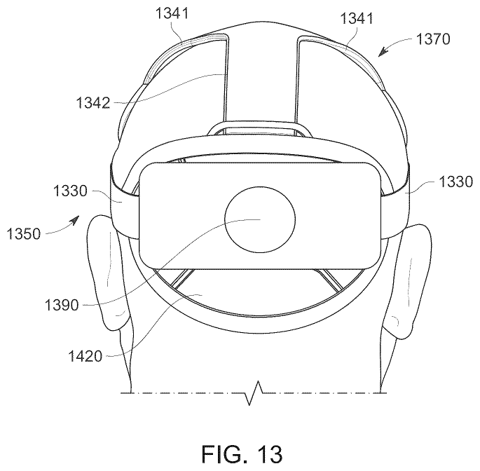

FIG. 13 is a posterior view of the head-mounted display system shown in FIG. 12.

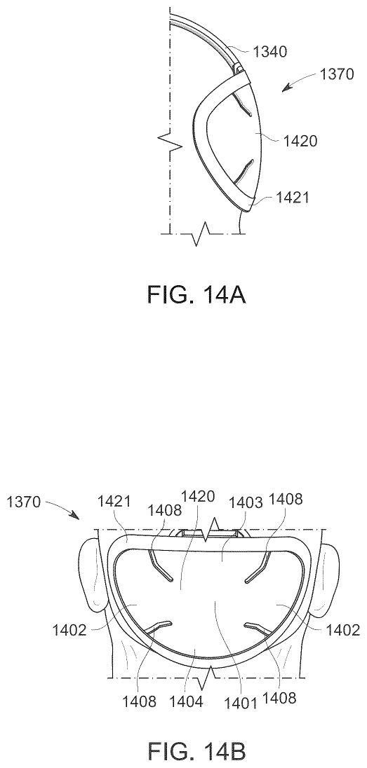

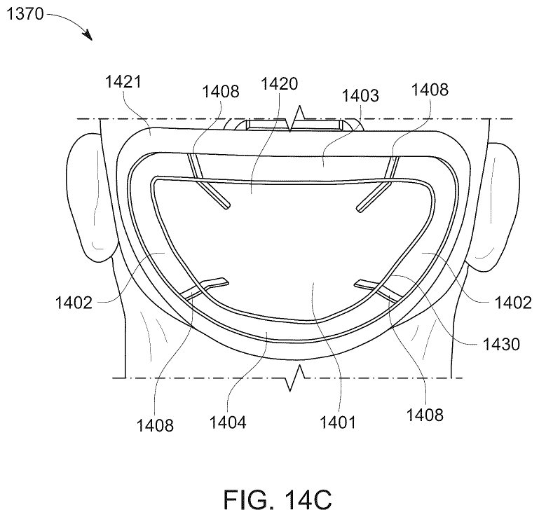

FIG. 14A is a lateral view of a posterior support pad assembly according to another example of the present technology.

FIG. 14B is a posterior view of the posterior support pad assembly shown in FIG. 14A.

FIG. 14C is a posterior view of a posterior support pad assembly according to another example of the present technology.

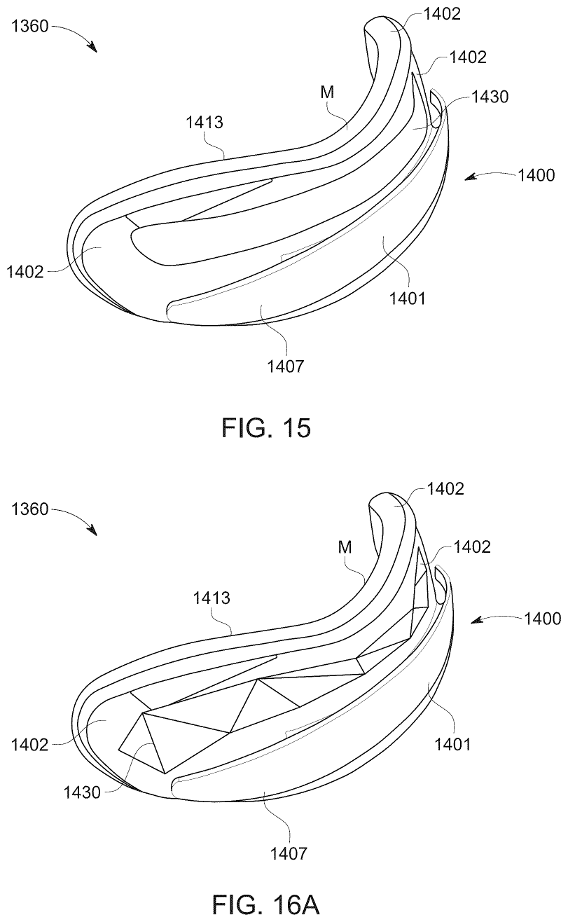

FIG. 15 is a perspective view of a forehead support according to another example of the present technology.

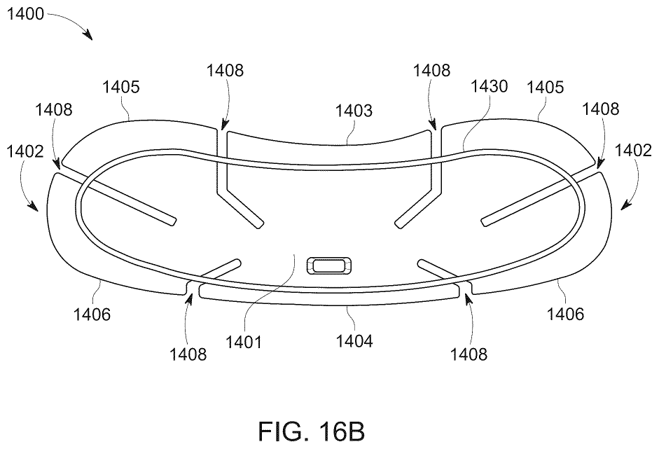

FIG. 16A is a perspective view of a forehead support according to another example of the present technology.

FIG. 16B is a front view of a frame of a forehead support according to another example of the present technology.

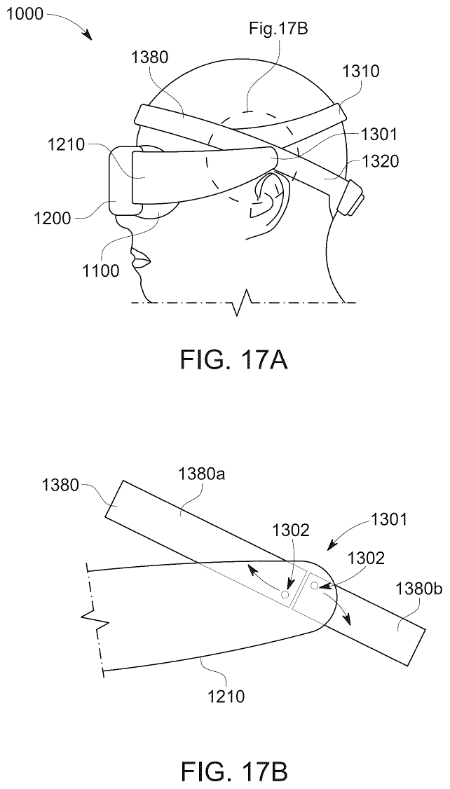

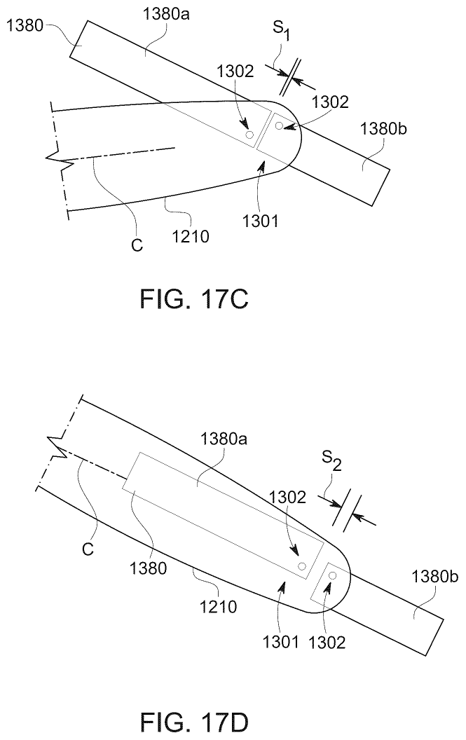

FIG. 17A shows a head-mounted display system according to another example of the present technology.

FIG. 17B shows portions of a positioning and stabilising structure of the head-mounted display system shown in FIG. 17A.

FIG. 17C shows portions of the positioning and stabilising structure shown in FIG. 17B when the head-mounted display unit is in a viewing position.

FIG. 17D shows portions of the positioning and stabilising structure shown in FIG. 17B when the head-mounted display unit is in a raised position.

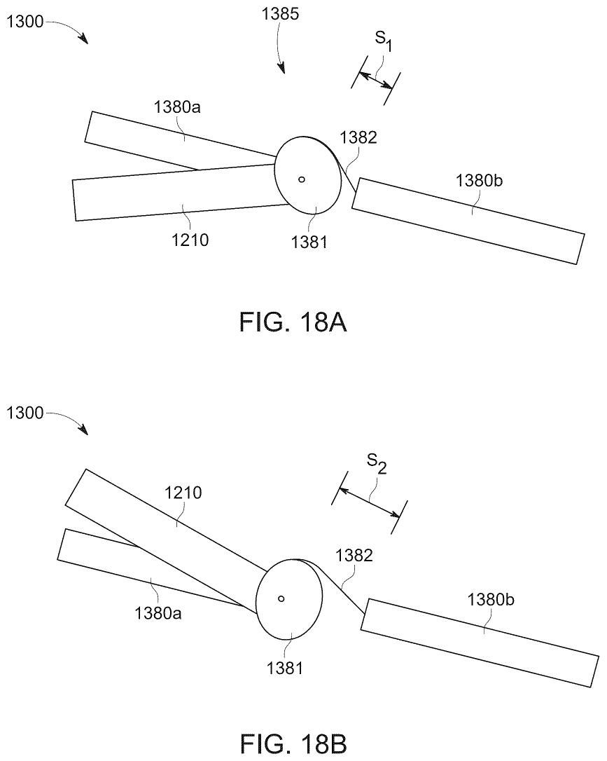

FIG. 18A shows a schematic view of a positioning and stabilising structure according to another example of the present technology when the head-mounted display unit is in a viewing position.

FIG. 18B shows the positioning and stabilising structure shown in FIG. 18A when the head-mounted display unit is in a raised position.

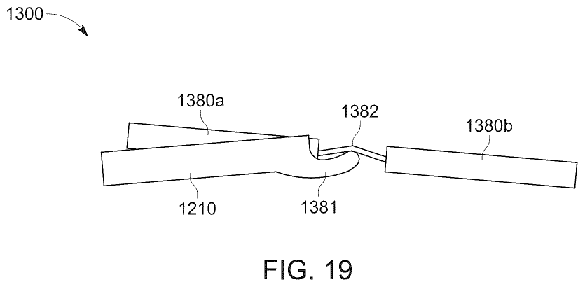

FIG. 19 shows a schematic view of a positioning and stabilising structure according to another example of the present technology when the head-mounted display unit is in a viewing position.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

In the following detailed description, reference is made to accompanying drawings which form a part of the detailed description. The illustrative embodiments described in the detailed description, depicted in the drawings and defined in the claims, are not intended to be limiting. Other embodiments may be utilised and other changes may be made without departing from the spirit or scope of the subject matter presented. It will be readily understood that the aspects of the present disclosure, as generally described herein and illustrated in the drawings can be arranged, substituted, combined, separated and designed in a wide variety of different configurations, all of which are contemplated in this disclosure.

### 5.1 Immersive Technologies

Immersive technologies may present a user with a combination of a virtual environment and the user's physical environment, or the real world. The user may interact with the resulting immersive or combined reality.

The device immerses the user by augmenting or replacing stimuli associated with one of the user's five senses with a virtual stimuli. Typically this is a virtual stimuli, although there could be additional stimuli that augment or replace stimuli associated with one of the additional four senses.

In some forms, a particular immersive technology may present a user with a combination of a virtual environment and the user's environment. At least a portion of the resulting environment may include a virtual environment. In some examples, the entire resulting environment may be a virtual environment (e.g., meaning the user's environment may be block from view or otherwise obstructed). In other forms, at least a portion of the user's physical environment may still be visually observable.

In some forms, the user may use different types of immersive technologies, which may include, but are not limited to, virtual reality (VR), augmented reality (AR), or mixed reality (MR). Each type of immersive technology may present the user with a different environment and/or a different way to interact with the environment.

In some forms, a display system may be used with each type of immersive technology. A display screen of the display system may provide a virtual environment component to the combination environment (i.e., the combination of the virtual and user's environments). In certain forms, the display screen may be an electronic screen.

In at least some types of immersive technologies (e.g., VR, AR, MR, etc.), positioning and stabilizing the electronic screen may be useful in operating a respective device. For example, the user may desire the electronic screen to be positioned close enough to their eyes to allow for easy viewing, but far enough away so as not to cause discomfort. Additionally, the electronic screen may need to be spaced far enough away so that users may simultaneously wear corrective lenses, like glasses. In addition, users may seek to maintain the orientation of the electronic screen relative to their eyes. In other words, users who walk, or otherwise move, while using these devices may not want the device to bounce or otherwise move on their head (e.g., particularly relative to their eyes), as this may cause dizziness and/or discomfort to the user. Therefore, these devices may be supported snuggly against the user's head in order to limit relative movement between the user's eyes and the device.

In one form, the present technology comprises a method for using a VR device comprising supporting the device on the user's head proximate to at least one of the user's eyes, and within the user's line of sight.

In certain examples of the present technology, a head-mounted display unit is supported in front of both of the user's eyes in order to block, obstruct, and/or limit ambient light from reaching the user's eyes.

Any features disclosed below in the context of a device configured for VR are to be understood as being applicable to devices configured for AR, unless the context clearly requires otherwise. Likewise features disclosed below in the context of a device configured for AR are to be understood as being applicable to devices configured for VR, unless the context clearly requires otherwise. For the avoidance of doubt, a feature disclosed in the context of a device that does not have a transparent display, through which the user can view the real world, is to be understood as being applicable to a device having such a transparent display unless the context clearly requires otherwise. Likewise a feature disclosed in the context of a device that has a transparent display, through which the real-world can be viewed, is to be understood to be applicable to a device in which the display is electronic and through which the real-world cannot be viewed directly through a transparent material.

### 5.2 Virtual Reality Display System

As shown in FIGS. 4A and 4B, a display apparatus, display system, display interface or head-mounted display system 1000 in accordance with one aspect of the present technology comprises the following functional aspects: an interfacing structure 1100, a head-mounted display unit 1200, and a positioning and stabilizing structure 1300. In some forms, a functional aspect may provide one or more physical components. In some forms, one or more physical components may provide one or more functional aspects. The head-mounted display unit 1200 may comprise a display. In use, the head-mounted display unit 1200 is arranged to be positioned proximate and anterior to the user's eyes, so as to allow the user to view the display.

In other aspects, the head-mounted display system 1000 may also include a display unit housing 1205, an optical lens 1240, a controller 1270, a speaker 1272, a power source 1274, and/or a control system 1276. In some examples, these may be integral pieces of the head-mounted display system 1000, while in other examples, these may be modular and incorporated into the head-mounted display system 1000 as desired by the user.

### 5.2.1 Head-Mounted Display Unit

The head-mounted display unit 1200 may include a structure for providing an observable output to a user. Specifically, the head-mounted display unit 1200 is arranged to be held (e.g., manually, by a positioning and stabilizing structure, etc.) in an operational position in front of a user's face.

In some examples, the head-mounted display unit 1200 may include a display screen 1220, a display unit housing 1205, an interfacing structure 1100, and/or an optical lens 1240. These components may be permanently assembled in a single head-mounted display unit 1200, or they may be separable and selectively connected by the user to form the head-mounted display unit 1200. Additionally, the display screen 1220, the display unit housing 1205, the interfacing structure 1100, and/or the optical lens 1240 may be included in the head-mounted display system 1000, but may not be part of the head-mounted display unit 1200.

### 5.2.1.1 Display Screen

Some forms of the head-mounted display unit 1200 include a display, for example a display screen—not shown in FIG. 4B, but provided within the display housing 1205. The display screen may include electrical components that provide an observable output to the user.

In one form of the present technology, a display screen provides an optical output observable by the user. The optical output allows the user to observe a virtual environment and/or a virtual object.

The display screen may be positioned proximate to the user's eyes, in order to allow the user to view the display screen. For example, the display screen may be positioned anterior to the user's eyes. The display screen can output computer generated images and/or a virtual environment.

### 5.2.1.2 Display Housing

In some forms of the present technology as shown in FIGS. 4A and 4B, a display unit housing 1205 provides a support structure for the display screen, in order to maintain a position of at least some of the components of the display screen relative to one another, and may additionally protect the display screen and/or other components of the head-mounted display unit 1200. The display unit housing 1205 may be constructed from a material suitable to provide protection from impact forces to the display screen. The display unit housing 1205 may also contact the user's face, and may be constructed from a biocompatible material suitable for limiting irritation to the user.

A display unit housing 1205 in accordance with some forms of the present technology may be constructed from a hard, rigid or semi-rigid material, such as plastic.

In certain forms, the rigid or semi-rigid material may be at least partially covered with a soft and/or flexible material (e.g., a textile, silicone, etc.). This may improve biocompatibility and/or user comfort because the at least a portion of the display unit housing 1205 that the user engages (e.g., grabs with their hands) includes the soft and/or flexible material.

A display unit housing 1205 in accordance with other forms of the present technology may be constructed from a soft, flexible, resilient material, such as silicone rubber.

### 5.2.1.3 Interfacing Structure

As shown in FIGS. 4A and 4B, some forms of the present technology include an interfacing structure 1100 is positioned and/or arranged in order to conform to a shape of a user's face, and may provide the user with added comfort while wearing and/or using the head-mounted display system 1000.

In some forms, the interfacing structure 1100 is coupled to a surface of the display unit housing 1205.

In some forms, the interfacing structure 1100 may extent at least partially around the display unit housing 1205, and may form a viewing opening. The viewing opening may at least partially receive the user's face in use. Specifically, the user's eyes may be received within the viewing opening formed by the interfacing structure 1100.

In some forms, the interfacing structure 1100 in accordance with the present technology may be constructed from a biocompatible material.

In some forms, the interfacing structure 1100 in accordance with the present technology may be constructed from a soft, flexible, and/or resilient material.

In certain forms, the interfacing structure 1100 in accordance with the present technology may be constructed from silicone rubber and/or foam.

In some forms, the interfacing structure 1100 may contact sensitive regions of the user's face, which may be locations of discomfort. The material forming the interfacing structure 1100 may cushion these sensitive regions, and limit user discomfort while wearing the head-mounted display system 1000.

In certain forms, these sensitive regions may include the user's forehead. Specifically, this may include the region of the user's head that is proximate to the frontal bone, like the Epicranius and/or the glabella. This region may be sensitive because there is limited natural cushioning from muscle and/or fat between the user's skin and the bone. Similarly, the ridge of the user's nose may also include little to no natural cushioning.

In some forms, the interfacing structure 1100 may comprise a single element. In some embodiments the interfacing structure 1100 may be designed for mass manufacture. For example, the interfacing structure 1100 may be designed to comfortably fit a wide range of different face shapes and sizes.

In some forms, the interfacing structure 1100 may include different elements that overlay different regions of the user's face. The different portions of the interfacing structure 1100 may be constructed from different materials, and provide the user with different textures and/or cushioning at different regions.

### 5.2.1.3.1 Light Shield

Some forms of the head-mounted display system 1000 may include a light shield that may be constructed from an opaque material and can block ambient light from reaching the user's eyes. The light shield may be part of the interfacing structure 1100 or may be a separate element. In some examples the interfacing structure 1100 may form a light shield by shielding the user's eyes from ambient light, in addition to providing a comfortable contacting portion for contact between the head-mounted display 1200 and the user's face. In some examples a light shield may be formed from multiple components working together to block ambient light.

### 5.2.1.3.1.1 Seal-Forming Structure

As shown in FIG. 4D, in one form of the present technology, the interfacing structure 1100 acts as a seal-forming structure, and provides a target seal-forming region. The target seal-forming region is a region on the seal-forming structure where sealing may occur. The region where sealing actually occurs—the actual sealing surface—may change within a given session, from day to day, and from user to user, depending on a range of factors including but not limited to, where the display unit housing 1205 is placed on the face, tension in the positioning and stabilizing structure 1300, and/or the shape of a user's face.

In one form the target seal-forming region is located on an outside surface of the interfacing structure 1100.

In some forms, the light shield may form the seal-forming structure and seal against the user's face.

In certain forms, the entire perimeter of the light shield or interfacing structure 1100 may seal against the user's skin, and can block ambient light from reaching an eye region. The eye region may be formed on regions of the Epicranius, the user's sphenoid, across the outer cheek region between the sphenoid to the left or right zygomatic arch, over the zygomatic arch, across the inner cheek region from the zygomatic arches towards the alar crests, and on the users' nasal ridge inferior to the sellion to enclose a portion of the users' face therebetween.

When acting as a seal-forming structure, the light shield or interfacing structure 1100 may contact sensitive areas the user's face, like the user's nasal ridge. This contact may entirely prevent the ingress of ambient light. Sealing around the entire perimeter of the display unit housing 1205 may improve performance of the head-mounted display system 1000. Additionally, biocompatible materials may be selected so that direct contact with the user's nasal ridge does not significantly reduce user comfort while wearing the head-mounted display system 1000.

In certain forms of the present technology, a system is provided comprising more than one interfacing structure 1100, each being configured to correspond to a different size and/or shape range. For example the system may comprise one form of interfacing structure 1100 suitable for a large sized head, but not a small sized head and another suitable for a small sized head, but not a large sized head. The different interfacing structures 1100 may be removable and replaceable so that different users with different sized heads may use the same head-mounted display system 1000.

In some forms, the seal-forming structure may be formed on regions of the Epicranius, the user's sphenoid, across the outer cheek region between the sphenoid to the left or right zygomatic arch, over the zygomatic arch, across the inner cheek region from the zygomatic arches towards the alar crests, and on the users' nasal ridge inferior to the sellion to enclose a portion of the users' face therebetween. This defined region may be an eye region.

In certain forms, this may seal around the user's eyes. The seal created by the seal-forming structure or interfacing structure 1100 may create a light seal, in order to limit ambient light from reaching the user's eyes.

### 5.2.1.3.2 Material Biocompatibility

Biocompatible materials are considered to be materials that undergo a full evaluation of their biological responses, relevant to their safety in use, according to ISO 10993-1 standard. The evaluation considers the nature and duration of anticipated contact with human tissues when in-use. In some forms of the present technology, the materials utilised in the positioning and stabilizing structure and interfacing structure may undergo at least some of the following biocompatibility tests: Cytotoxicity-Elution Test (MeM Extract): ANSI/AAMI/ISO 10993-5; Skin Sensitisation: ISO 10993-10; Irritation: ISO 10993-10; Genotoxicity-Bacterial Mutagenicity Test: ISO 10993-3; Implantation: ISO 10993-6.

### 5.2.2 Positioning and Stabilizing Structure

As shown in FIGS. 4A and 4B, the display screen 1220 and/or the display unit housing 1205 of the head-mounted display system 1000 of the present technology may be held in position in use by the positioning and stabilizing structure 1300.

To hold the display screen 1220 and/or the display unit housing 1205 in its correct operational position, the positioning and stabilizing structure 1300 is ideally comfortable against the user's head in order to accommodate the induced loading from the weight of the display unit in a manner that minimise facial markings and/or pain from prolonged use. There is also need to allow for a universal fit without trading off comfort, usability and cost of manufacture. The design criteria may include adjustability over a predetermined range with low-touch simple set up solutions that have a low dexterity threshold. Further considerations include catering for the dynamic environment in which the head-mounted display system 1000 may be used. As part of the immersive experience of a virtual environment, users may communicate, i.e. speak, while using the head-mounted display system 1000. In this way, the jaw or mandible of the user may move relative to other bones of the skull. Additionally, the whole head may move during the course of a period of use of the head-mounted display system 1000. For example, movement of a user's upper body, and in some cases lower body, and in particular, movement of the head relative to the upper and lower body.

In one form the positioning and stabilizing structure 1300 provides a retention force to overcome the effect of the gravitational force on the display screen 1220 and/or the display unit housing 1205.

In one form of the present technology, a positioning and stabilizing structure 1300 is provided that is configured in a manner consistent with being comfortably worn by a user. In one example the positioning and stabilizing structure 1300 has a low profile, or cross-sectional thickness, to reduce the perceived or actual bulk of the apparatus. In one example, the positioning and stabilizing structure 1300 comprises at least one strap having a rectangular cross-section. In one example the positioning and stabilizing structure 1300 comprises at least one flat strap.

In one form of the present technology, a positioning and stabilizing structure 1300 is provided that is configured so as not to be too large and bulky to prevent the user from comfortably moving their head from side to side.

In one form of the present technology, a positioning and stabilizing structure 1300 comprises a strap constructed from a laminate of a textile user-contacting layer, a foam inner layer and a textile outer layer. In one form, the foam is porous to allow moisture, (e.g., sweat), to pass through the strap. In one form, a skin contacting layer of the strap is formed from a material that helps wick moisture away from the user's face. In one form, the textile outer layer comprises loop material to engage with a hook material portion.

In certain forms of the present technology, a positioning and stabilizing structure 1300 comprises a strap that is extensible, e.g. resiliently extensible. For example the strap may be configured in use to be in tension, and to direct a force to draw the display screen 1220 and/or the display unit housing 1205 toward a portion of a user's face, particularly proximate to the user's eyes and in line with their field of vision. In an example the strap may be configured as a tie.

In one form of the present technology, the positioning and stabilizing structure 1300 comprises a first tie, the first tie being constructed and arranged so that in use at least a portion of an inferior edge thereof passes superior to an otobasion superior of the user's head and overlays a portion of a parietal bone without overlaying the occipital bone.

In one form of the present technology, the positioning and stabilizing structure 1300 includes a second tie, the second tie being constructed and arranged so that in use at least a portion of a superior edge thereof passes inferior to an otobasion inferior of the user's head and overlays or lies inferior to the occipital bone of the user's head.

In one form of the present technology, the positioning and stabilizing structure 1300 includes a third tie that is constructed and arranged to interconnect the first tie and the second tie to reduce a tendency of the first tie and the second tie to move apart from one another.

In certain forms of the present technology, a positioning and stabilizing structure 1300 comprises a strap that is bendable and e.g. non-rigid. An advantage of this aspect is that the strap is more comfortable against a user's head.

In certain forms of the present technology, a positioning and stabilizing structure 1300 comprises a strap constructed to be breathable to allow moisture vapour to be transmitted through the strap,

In certain forms of the present technology, a system is provided comprising more than one positioning and stabilizing structure 1300, each being configured to provide a retaining force to correspond to a different size and/or shape range. For example the system may comprise one form of positioning and stabilizing structure 1300 suitable for a large sized head, but not a small sized head, and another. Suitable for a small sized head, but not a large sized head.

In some forms, the positioning and stabilizing structure 1300 may include cushioning material (e.g., a foam pad) for contacting the user's skin. The cushioning material may provide added wearability to the positioning and stabilizing structure 1300, particularly if positioning and stabilizing structure 1300 is constructed from a rigid or semi-rigid material.

### 5.2.2.1 Temporal Connectors

As shown in FIG. 4C, some forms of the head-mounted display system 1000 or positioning and stabilizing structure 1300 include temporal connectors 1250, each of which may overlay a respective one of the user's temporal bones in use. A portion of the temporal connectors 1250, in-use, are in contact with a region of the user's head proximal to the otobasion superior, i.e. above each of the user's ears. In some examples, temporal connectors are strap portions of a positioning and stabilising structure 1300. In other examples, temporal connectors are arms of a head-mounted display unit 1200. In some examples a temporal connector of a head-mounted display system 1000 may be formed partially by a strap portion (e.g. a lateral strap portion 1330) of a positioning and stabilising structure 1300 and partially by an arm 1210 of a head-mounted display unit 1200.

The temporal connectors 1250 may be lateral portions of the positioning and stabilizing structure 1300, as each temporal connector 1250 is positioned on either the left or the right side of the user's head.

In some forms, the temporal connectors 1250 may extend in an anterior-posterior direction, and may be substantially parallel to the sagittal plane.

In some forms, the temporal connectors 1250 may be coupled to the display unit housing 1205. For example, the temporal connectors 1250 may be connected to lateral sides of the display unit housing 1205. For example, each temporal connector 1250 may be coupled to a respective one of the lateral left face 1234 and the lateral right face 1236.

In certain forms, the temporal connectors 1250 may be pivotally connected to the display unit housing 1205, and may provide relative rotation between each temporal connector 1250, and the display unit housing 1205.

In certain forms, the temporal connectors 1250 may be removably connected to the display unit housing 1205 (e.g., via a magnet, a mechanical fastener, hook and loop material, etc.).

In some forms, the temporal connectors 1250 may be arranged in-use to run generally along or parallel to the Frankfort Horizontal plane of the head and superior to the zygomatic bone (e.g., above the user's cheek bone).

In some forms, the temporal connectors 1250 may be positioned against the user's head similar to arms of eye-glasses, and be positioned more superior than the anti-helix of each respective ear.

In some forms, the temporal connectors 1250 may have a generally elongate and flat configuration. In other words, each temporal connector 1250 is far longer and wider (direction from top to bottom in the paper plane) than thick (direction into the paper plane).

In some forms, the temporal connectors 1250 may each have a three-dimensional shape which has curvature in all three axes (X, Y and Z). Although the thickness of each temporal connector 1250 may be substantially uniform, its height varies throughout its length. The purpose of the shape and dimension of each temporal connector 1250 is to conform closely to the head of the user in order to remain unobtrusive and maintain a low profile (e.g., not appear overly bulky).

In some forms, the temporal connectors 1250 may be constructed from a rigid or semi-rigid material, which may include plastic, hytrel (thermoplastic polyester elastomer), or another similar material. The rigid or semi-rigid material may be self-supporting and/or able to hold its shape without being worn. This can make it more intuitive or obvious for users to understand how to use the positioning and stabilizing structure 1300 and may contrast with a positioning and stabilizing structure 1300 that is entirely floppy and does not retain a shape. Maintaining the temporal connectors 1250 in the in-use state prior to use may prevent or limit distortion whilst the user is donning the positioning and stabilizing structure 1300 and allow a user to quickly fit or wear the head-mounted display system 1000.

In certain forms, the temporal connectors 1250 may be rigidizers, which may allow for a more effective (e.g., direct) translation of tension through the temporal connectors 1250 because rigidizers limit the magnitude of elongation or deformation of the arm while in-use.

In certain forms, the positioning and stabilizing structure 1300 may be designed so that the positioning and stabilizing structure 1300 springs ‘out of the box’ and generally into its in-use configuration. In addition, the positioning and stabilizing structure 1300 may be arranged to hold its in-use shape once out of the box (e.g., because rigidizers may be formed to maintain the shape of some or part of the positioning and stabilizing structure 1300). Advantageously, the orientation of the positioning and stabilizing structure 1300 is made clear to the user as the shape of the positioning and stabilizing structure 1300 is generally curved much like the rear portion of the user's head. That is, the positioning and stabilizing structure 1300 is generally dome shaped.

In certain forms, a flexible and/or resilient material may be disposed around the rigid or semi-rigid material of the temporal connectors 1250. The flexible material may be more comfortable against the user's head, in order to improve wearability and provide soft contact with the user's face. In one form, the flexible material is a textile sleeve at is permanently or removably coupled to each temporal connector 1250.

In one form, a textile may be over-moulded onto at least one side of the rigidizer. In one form, the rigidizer may be formed separately to the resilient component and then a sock of user contacting material (e.g., Breath-O-Prene™) may be wrapped or slid over the rigidizer. In alternative forms, the user contacting material may be provided to the rigidizer by adhesive, ultrasonic welding, sewing, hook and loop material, and/or stud connectors.

In some forms, the user contacting material may be on both sides of the rigidizer, or alternatively may only be on the user contacting side (e.g., the user contacting side) of the rigidizer to reduce bulk and cost of materials.

In some forms, the temporal connectors 1250 are constructed from a flexible material (e.g., a textile), which may be comfortable against the user's skin, and may not require an added layer to increase comfort.

### 5.2.2.2 Posterior Support Portion

As shown in FIG. 4C, some forms of the positioning and stabilizing structure 1300 may include a posterior support portion 1350 for assisting in supporting the display screen 1220 and/or the display unit housing 1205 (shown in FIG. 4B) proximate to the user's eyes. The posterior support portion 1350 may assist in anchoring the display screen and/or the display unit housing 1205 to the user's head in order to appropriately orient the display screen proximate to the user's eyes.

In some forms, the posterior support portion 1350 may be coupled to the display unit housing 1205 via the temporal connectors 1250.

In certain forms, the temporal connectors 1250 may be directly coupled to the display unit housing 1205 and to the posterior support portion 1350.

In some forms, the posterior support portion 1350 may have a three-dimensional contour curve to fit to the shape of a user's head. For example, the three-dimensional shape of the posterior support portion 1350 may have a generally round three-dimensional shape adapted to overlay a portion of the parietal bone and the occipital bone of the user's head, in use.

In some forms, the posterior support portion 1350 may be a posterior portion of the positioning and stabilizing structure 1300. The posterior support portion 1350 may provide an anchoring force directed at least partially in the anterior direction.

In certain forms, the posterior support portion 1350 is the inferior-most portion of the positioning and stabilizing structure 1300. For example, the posterior support portion 1350 may contact a region of the user's head between the occipital bone and the trapezius muscle. The posterior support portion 1350 may hook against an inferior edge of the occipital bone (e.g., the occiput). The posterior support portion 1350 may provide a force directed in the superior direction and/or the anterior direction in order to maintain contact with the user's occiput.

In certain forms, the posterior support portion 1350 is the inferior-most portion of the entire head-mounted display system 1000. For example, the posterior support portion 1350 may be positioned at the base of the user's neck (e.g., overlaying the occipital bone and the trapezius muscle more inferior than the user's eyes) so that the posterior support portion 1350 is more inferior than the display screen 1220 and/or the display unit housing 1205.

In some forms, the posterior support portion 1350 may include a padded material, which may contact the user's head (e.g., overlaying the region between the comfort to the user, and limit marks caused by the posterior support portion 1350 pulling against the user's head.

### 5.2.2.3 Forehead Support

Some forms of the positioning and stabilizing structure 1300 may include a forehead support or frontal support portion 1360 configured to contact the user's head superior to the user's eyes, while in use. The positioning and stabilising structure 1300 shown in FIG. 5B includes a forehead support 1360. In some examples the positioning and stabilising structure 1300 shown in FIG. 4A may include a forehead support 1360. The forehead support 1360 may overlay the frontal bone of the user's head. In certain forms, the forehead support 1360 may also be more superior than the sphenoid bones and/or the temporal bones. This may also position the forehead support 1360 more superior than the user's eyebrows.

In some forms, the forehead support 1360 may be an anterior portion of the positioning and stabilizing structure 1300, and may be disposed more anterior on the user's head than any other portion of the positioning and stabilizing structure 1300. The posterior support portion 1350 may provide a force directed at least partially in the posterior direction.

In some forms, the forehead support 1360 may include a cushioning material (e.g., textile, foam, silicone, etc.) that may contact the user, and may help to limit marks caused by the straps of the positioning and stabilizing structure 1300. The forehead support 1360 and the interfacing structure 1100 may work together in order to provide comfort to the user.

In some forms, the forehead support 1360 may be separate from the display unit housing 1205, and may contact the user's head at a different location (e.g., more superior) than the display unit housing 1205.

In some forms, the forehead support 1360 can be adjusted to allow the positioning and stabilizing structure 1300 to accommodate the shape and/or configuration of a user's face.

In some forms, the temporal connectors 1250 may be coupled to the forehead support 1360 (e.g., on lateral sides of the forehead support 1360). The temporal connectors 1250 may extend at least partially in the inferior direction in order to couple to the posterior support portion 1350.

In certain forms, the positioning and stabilizing structure 1300 may include multiple pairs of temporal connectors 1250. For example, one pair of temporal connectors 1250 may be coupled to the forehead support 1360, and one pair of temporal connectors 1250 may be coupled to the display unit housing 1205.

In some forms, the forehead support 1360 can be presented at an angle which is generally parallel to the user's forehead to provide improved comfort to the user. For example, the forehead support 1360 may position the user in an orientation that overlays the frontal bone, and is substantially parallel to the coronal plane. Positioning the forehead support substantially parallel to the coronal plane can reduce the likelihood of pressure sores which may result from an uneven presentation.

In some forms, the forehead support 1360 may be offset from a rear support or posterior support portion that contacts a posterior region of the user's head (e.g., an area overlaying the occipital bone and the trapezius muscle). In other words, an axis along a rear strap would not intersect the forehead support 1360, which may be disposed more inferior and anterior than the axis along the rear strap. The resulting offset between the forehead support 1360 and the rear strap may create moments that oppose the weight force of the display screen 1220 and/or the display unit housing 1205. A larger offset may create a larger moment, and therefore more assistance in maintaining a proper position of the display screen 1220 and/or the display unit housing 1205. The offset may be increased by moving the forehead support 1360 closer to the user's eyes (e.g., more anterior and inferior along the user's head), and/or increasing the angle of the rear strap so that it is more vertical.

### 5.2.2.4 Adjustable Straps

As shown in FIG. 4C, portions of the positioning and stabilizing structure 1300 may be adjustable, in order to impart a selective tensile force on the display screen 1220 and/or the display unit housing 1205 in order to secure a position of the display screen 1220 and/or the display unit housing 1205.

In some forms, the display unit housing 1205 may include at least one loop or eyelet 1254 (as shown in FIG. 4B), and at least one of the temporal connectors 1250 may be threaded through that loop, and doubled back on itself. The length of the temporal connector 1250 threaded through the respective eyelet 1254 may be selected by the user in order to adjust the tensile force provided by the positioning and stabilizing structure 1300. For example, threading a greater length of the temporal connector 1250 through the eyelet 1254 may supply a greater tensile force.

In some forms, at least one of the temporal connectors 1250 may include an adjustment portion 1256 and a receiving portion 1258 (as shown in FIG. 4C). The adjustment portion 1256 may be positioned through the eyelet 1254 on the display unit housing 1205, and may be coupled to the receiving portion 1258 (e.g., by doubling back on itself). The adjustment portion 1256 may include a hook material, and the receiving portion 1258 may include a loop material (or vice versa), so that the adjustment portion 1256 may be removably held in the desired position. In some examples, the hook material and the loop material may be Velcro.

In certain forms, adjusting the position of the adjustment portion 1256 relative to the receiving portion 1258 may apply a posterior force to the display screen 1220 and/or the display unit housing 1205, and increase or decrease a sealing force of the light shield against the user's head (e.g., when the light shield acts as a seal-forming structure).

In certain forms, the adjustment portion 1256 may be constructed from a flexible and/or resilient material, which may conform to a shape of the user's head and/or may allow the adjustment portion to be threaded through the eyelet 1254. For example, the adjustment portion(s) 1256 may be constructed from an elastic textile, which may provide an elastic, tensile force. The remainder of the temporal connectors 1250 may be constructed from the rigid or semi-rigid material described above (although it is contemplated that additional sections of the temporal connectors 1250 may also be constructed from a flexible material).

### 5.2.2.4.1 Top Strap

In some forms, the positioning and stabilizing structure 1300 may include a top strap portion, which may overlay a superior region of the user's head. The head-mounted display system 1000 shown in FIG. 1A has a top strap portion, for example.

In some forms, the top strap portion may extend between an anterior portion of the head-mounted display system 1000 and a posterior region of the head-mounted display system 1000.

In some forms, the top strap portion may be constructed from a flexible material, and may be configured to compliment the shape of the user's head.

In certain forms, the top strap portion may be connected to the display unit housing 1205. For example, the top strap portion may be coupled to the superior face 1230. The top strap portion may also be coupled to the display unit housing 1205 proximate to a posterior end of the display unit housing 1205.

In certain forms, the top strap portion may be coupled to the forehead support 1360. For example, the top strap portion may be coupled to the forehead support 1360 proximate to a superior edge. The top strap portion may be connected to the display unit housing 1205 through the forehead support 1360.

In some forms, the top strap portion may be connected to the posterior support portion 1350. For example, the top strap portion may be connected proximate to a superior edge of the posterior support portion 1350.

In some forms, the top strap portion may overlay the frontal bone and the parietal bone of the user's head.

In certain forms, the top strap portion may extend along the sagittal plane as it extends between the anterior and posterior portions of the head-mounted display system 1000.

In certain forms, the top strap portion may apply a tensile force oriented at least partially in the superior direction, which may oppose the force of gravity.

In certain forms, the top strap portion may apply a tensile force oriented at least partially in the posterior direction, which may pull the interfacing structure 1100 toward the user's face (and supply a portion of the sealing force when the light shield acts as a seal-forming structure).

In some forms, the top strap portion may be adjustable in order to impart a selective tensile force on the display screen 1220 and/or the display unit housing 1205 in order to secure a position of the display screen 1220 and/or the display unit housing 1205.

In certain forms, the display unit housing 1205 and/or the forehead support 1360 (as the case may be) may include at least one loop or eyelet 1254, and the top strap portion may be threaded through that eyelet 1254, and doubled back on itself. The length of the top strap portion threaded through the eyelet 1254 may be selected by the user in order to adjust the tensile force provided by the positioning and stabilizing structure 1300. For example, threading a greater length of the top strap portion through the eyelet 1254 may supply a greater tensile force.

In some forms, the top strap portion may include an adjustment portion and a receiving portion. The adjustment portion may be positioned through the eyelet 1254, and may be coupled to the receiving portion (e.g., by doubling back on itself). The adjustment portion may include a hook material, and the receiving portion may include a loop material (or vice versa), so that the adjustment portion may be removably held in the desired position. In some examples, the hook material and the loop material may be Velcro.

### 5.3 Augmented Reality Display Interface

As shown in FIGS. 5A and 5B, a display apparatus or head-mounted display system 1000 in accordance with one aspect of the present technology comprises the following functional aspects: a display screen 1220, a display unit housing 1205, and a positioning and stabilizing structure 1300. In some forms, a functional aspect may provide one or more physical components. In some forms, one or more physical components may provide one or more functional aspects. In use, the display screen 1220 is arranged to be positioned proximate and anterior to the user's eyes, so as to allow the user to view the display screen 1220.

In other aspects, the head-mounted display system 1000 may also include an interfacing structure 1100, a controller 1270, a speaker 1272, a power source 1274, and/or a control system 1276. In some examples, these may be integral pieces of the head-mounted display system 1000, while in other examples, these may be modular and incorporated into the head-mounted display system 1000 as desired by the user.

### 5.3.1 Display Unit

The head-mounted display unit 1200 may include a structure for providing an observable output to a user. Specifically, the head-mounted display unit 1200 is arranged to be held (e.g., manually, by a positioning and stabilizing structure, etc.) in an operational position in front of a user's face.

In some examples, the head-mounted display unit 1200 may include a display screen 1220, a display unit housing 1205, and/or an interfacing structure 1100. These components may be integrally formed in a single head-mounted display unit 1200, or they may be separable and selectively connected by the user to form the head-mounted display unit 1200. Additionally, the display screen 1220, the display unit housing 1205, and/or the interfacing structure 1100 may be included in the head-mounted display system 1000, but may not be part of the head-mounted display unit 1200.

### 5.3.1.1 Display Screen

As shown in FIG. 5A, some forms of the head-mounted display unit 1200 include a display screen 1220. The display screen 1220 may include electrical components that provide an observable output to the user.

In one form of the present technology shown in FIG. 5A and FIG. 5B, a display screen 1220 provides an optical output observable by the user. The optical output allows the user to observe a virtual environment and/or a virtual object.

The display screen 1220 may be positioned proximate to the user's eyes, in order to allow the user to view the display screen 1220. For example, the display screen 1220 maybe positioned anterior to the user's eyes. The display screen 1220 can display computer generated images that can be view by the user in order to augment the user's physical environment (e.g., the computer generated images may appear as though they are present in the user's physical environment).

In certain forms, particularly when using the display screen 1220 in an AR or MR environment, the display screen 1220 may be turned off while the user continues to wear the display screen 1220 and interact with the physical environment. This may allow the user to selectively choose when to receive the virtual stimulation, and when to observe only the physical environment.

In certain forms, the display screen 1220 may be transparent (or translucent). For example, the display screen 1220 may be glass, so the user can see through the display screen 1220. This may be particularly beneficial in AR or MR applications, so that the user can continue to see the physical environment.

### 5.3.1.2 Display Housing

In some forms of the present technology as shown in FIGS. 5A and 5B, a display unit housing 1205 provides a support structure for the display screen 1220, in order to maintain a position of at least some of the components of the display screen 1220 relative to one another, and may additionally protect the display screen 1220 and/or other components of the head-mounted display unit 1200. The display unit housing 1205 may be constructed from a material suitable to provide protection from impact forces to the display screen 1220. The display unit housing 1205 may also contact the user's face, and may be constructed from a biocompatible material suitable for limiting irritation to the user.

A display unit housing 1205 in accordance with some forms of the present technology may be constructed from a hard, rigid or semi-rigid material, such as plastic.

In certain forms, the rigid or semi-rigid material may be at least partially covered with a soft and/or flexible material (e.g., a textile, silicone, etc.). This may improve biocompatibility and/or user comfort because the at least a portion of the display unit housing 1205 that the user engages (e.g., grabs with their hands) includes the soft and/or flexible material.

A display unit housing 1205 in accordance with other forms of the present technology may be constructed from a soft, flexible, resilient material, such as silicone rubber.

In some forms, the display screen 1220 may project at least partially out of the display unit housing 1205. For example, unlike in a VR head-mounted display system 1000, the display screen 1220 in an AR (or MR) head-mounted display system 1000 may not be completely enclosed by the by the display unit housing 1205. The user may be able to directly view the display screen 1220, and may be able to look through the display screen 1220 (e.g., if the display screen 1220 is transparent or translucent).

In certain forms, the display unit housing 1205 may support sensors or other electronics described below. The display unit housing 1205 may provide protection to the electronics without substantially obstructing the user's view of the display screen 1220.

### 5.3.1.3 Interface Structure

As shown in FIGS. 5A and 5B, some forms of the present technology include an interfacing structure 1100 (also identified as “interface”, “user interface”, “interface structure” or the like) is positioned and/or arranged in order to conform to a shape of a user's face, and may provide the user with added comfort while wearing and/or using the head-mounted display system 1000.

In some forms, the interfacing structure 1100 is coupled to a surface of the display unit housing 1205.

In some forms, the interfacing structure 1100 in accordance with the present technology may be constructed from a biocompatible material.

In some forms, the interfacing structure 1100 in accordance with the present technology may be constructed from a soft, flexible, and/or resilient material.

In certain forms, the interfacing structure 1100 in accordance with the present technology may be constructed from silicone rubber and/or foam.

In some forms, the interfacing structure 1100 may contact sensitive regions of the user's face, which may be locations of discomfort. The material forming the interfacing structure 1100 may cushion these sensitive regions, and limit user discomfort while wearing the head-mounted display system 1000.

In certain forms, these sensitive regions may include the user's forehead. Specifically, this may include the region of the user's head that is proximate to the frontal bone, like the Epicranius and/or the glabella. This region may be sensitive because there is limited natural cushioning from muscle and/or fat between the user's skin and the bone. Similarly, the ridge of the user's nose may also include little to no natural cushioning.

In some forms, the interfacing structure 1100 can comprise a single element. In some embodiments the interfacing structure 1100 may be designed for mass manufacture. For example, the interfacing structure 1100 can be designed to comfortably fit a wide range of different face shapes and sizes.

In some forms, the interfacing structure 1100 may include different elements that overlay different regions of the user's face. The different portions of the interfacing structure 1100 may be constructed from different materials, and provide the user with different textures and/or cushioning at different regions.

In some forms, the interface structure 1100 may include nasal pads (e.g., as used in eye-glasses) that may contact the lateral sides of the user's nose. The nasal pads may apply light pressure to the user's nose to maintain the position of the head-mounted display system 1000, but may not apply a force that causes significant discomfort (e.g., the nasal pads may not receive a posterior directed tensile force).

### 5.3.2 Positioning and Stabilizing Structure

As shown in FIGS. 5A to 5B, the display screen 1220 and/or the display unit housing 1205 of the head-mounted display system 1000 of the present technology may be held in position in use by the positioning and stabilizing structure 1300.

To hold the display screen 1220 and/or the display unit housing 1205 in its correct operational position, the positioning and stabilizing structure 1300 is ideally comfortable against the user's head in order to accommodate the induced loading from the weight of the display unit in a manner that minimise facial markings and/or pain from prolonged use. There is also need to allow for a universal fit without trading off comfort, usability and cost of manufacture. The design criteria may include adjustability over a predetermined range with low-touch simple set up solutions that have a low dexterity threshold. Further considerations include catering for the dynamic environment in which the head-mounted display system 1000 may be used. As part of the immersive experience of a virtual environment, users may communicate, i.e. speak, while using the head-mounted display system 1000. In this way, the jaw or mandible of the user may move relative to other bones of the skull. Additionally, the whole head may move during the course of a period of use of the head-mounted display system 1000. For example, movement of a user's upper body, and in some cases lower body, and in particular, movement of the head relative to the upper and lower body.

In one form the positioning and stabilizing structure 1300 provides a retention force to overcome the effect of the gravitational force on the display screen 1220 and/or the display unit housing 1205.

In one form of the present technology, a positioning and stabilizing structure 1300 is provided that is configured in a manner consistent with being comfortably worn by a user. In one example the positioning and stabilizing structure 1300 has a low profile, or cross-sectional thickness, to reduce the perceived or actual bulk of the apparatus. In one example, the positioning and stabilizing structure 1300 comprises at least one strap having a rectangular cross-section. In one example the positioning and stabilizing structure 1300 comprises at least one flat strap.

In one form of the present technology, a positioning and stabilizing structure 1300 is provided that is configured so as not to be too large and bulky to prevent the user from comfortably moving their head from side to side.

In one form of the present technology, a positioning and stabilizing structure 1300 comprises a strap constructed from a laminate of a textile user-contacting layer, a foam inner layer and a textile outer layer. In one form, the foam is porous to allow moisture, (e.g., sweat), to pass through the strap. In one form, a skin contacting layer of the strap is formed from a material that helps wick moisture away from the user's face. In one form, the textile outer layer comprises loop material to engage with a hook material portion.

In certain forms of the present technology, a positioning and stabilizing structure 1300 comprises a strap that is extensible, e.g. resiliently extensible. For example the strap may be configured in use to be in tension, and to direct a force to draw the display screen 1220 and/or the display unit housing 1205 toward a portion of a user's face, particularly proximate to the user's eyes and in line with their field of vision. In an example the strap may be configured as a tie.

In one form of the present technology, the positioning and stabilizing structure 1300 comprises a first tie, the first tie being constructed and arranged so that in use at least a portion of an inferior edge thereof passes superior to an otobasion superior of the user's head and overlays a portion of a parietal bone without overlaying the occipital bone.

In one form of the present technology, the positioning and stabilizing structure 1300 includes a second tie, the second tie being constructed and arranged so that in use at least a portion of a superior edge thereof passes inferior to an otobasion inferior of the user's head and overlays or lies inferior to the occipital bone of the user's head.

In one form of the present technology, the positioning and stabilizing structure 1300 includes a third tie that is constructed and arranged to interconnect the first tie and the second tie to reduce a tendency of the first tie and the second tie to move apart from one another.

In certain forms of the present technology, a positioning and stabilizing structure 1300 comprises a strap that is bendable and e.g. non-rigid. An advantage of this aspect is that the strap is more comfortable against a user's head.

In certain forms of the present technology, a positioning and stabilizing structure 1300 comprises a strap constructed to be breathable to allow moisture vapour to be transmitted through the strap,

In certain forms of the present technology, a system is provided comprising more than one positioning and stabilizing structure 1300, each being configured to provide a retaining force to correspond to a different size and/or shape range. For example the system may comprise one form of positioning and stabilizing structure 1300 suitable for a large sized head, but not a small sized head, and another. Suitable for a small sized head, but not a large sized head.

In some forms, the positioning and stabilizing structure 1300 may include cushioning material (e.g., a foam pad) for contacting the user's skin. The cushioning material may provide added wearability to the positioning and stabilizing structure 1300, particularly if positioning and stabilizing structure 1300 is constructed from a rigid or semi-rigid material.

### 5.3.2.1 Temporal Connectors

As shown in FIG. 5B, some forms of the positioning and stabilizing structure 1300 include temporal connectors 1250, each of which may overlay a respective one of the user's temporal bones in use. A portion of the temporal connectors 1250, in-use, are in contact with a region of the user's head proximal to the otobasion superior, i.e. above each of the user's ears.

The temporal connectors 1250 may be lateral portions of the positioning and stabilizing structure 1300, as each temporal connector 1250 is positioned on either the left or the right side of the user's head.

In some forms, the temporal connectors 1250 may extend in an anterior-posterior direction, and may be substantially parallel to the sagittal plane.

In some forms, the temporal connectors 1250 may be coupled to the display unit housing 1205. For example, the temporal connectors 1250 may be connected to lateral sides of the display unit housing 1205.

In some forms, the temporal connectors 1250 may be arranged in-use to run generally along or parallel to the Frankfort Horizontal plane of the head and superior to the zygomatic bone (e.g., above the user's cheek bone).

In some forms, the temporal connectors 1250 may be positioned against the user's head similar to arms of eye-glasses, and be positioned more superior than the anti-helix of each respective ear.

In some forms, the temporal connectors 1250 may have a generally elongate and flat configuration. In other words, each temporal connector 1250 is far longer and wider (direction from top to bottom in the paper plane) than thick (direction into the paper plane).

In some forms, the temporal connectors 1250 may each have a three-dimensional shape which has curvature in all three axes (X, Y and Z). Although the thickness of each temporal connector 1250 may be substantially uniform, its height varies throughout its length. The purpose of the shape and dimension of each temporal connector 1250 is to conform closely to the head of the user in order to remain unobtrusive and maintain a low profile (e.g., not appear overly bulky).

In some forms, the temporal connectors 1250 may be constructed from a rigid or semi-rigid material, which may include plastic, Hytrel (thermoplastic polyester elastomer), or another similar material. The rigid or semi-rigid material may be self-supporting and/or able to hold its shape without being worn. This can make it more intuitive or obvious for users to understand how to use the positioning and stabilizing structure 1300 and may contrast with a positioning and stabilizing structure 1300 that is entirely floppy and does not retain a shape. Maintaining the temporal connectors 1250 in the in-use state prior to use may prevent or limit distortion whilst the user is donning the positioning and stabilizing structure 1300 and allow a user to quickly fit or wear the head-mounted display system 1000.

In certain forms, the temporal connectors 1250 may be rigidizers, which may allow for a more effective (e.g., direct) translation of tension through the temporal connectors 1250 because rigidizers limit the magnitude of elongation or deformation of the arm while in-use.

In certain forms, the positioning and stabilizing structure 1300 may be designed so that the positioning and stabilizing structure 1300 springs ‘out of the box’ and generally into its in-use configuration. In addition, the positioning and stabilizing structure 1300 may be arranged to hold its in-use shape once out of the box (e.g., because rigidizers may be formed to maintain the shape of some or part of the positioning and stabilizing structure 1300). Advantageously, the orientation of the positioning and stabilizing structure 1300 is made clear to the user as the shape of the positioning and stabilizing structure 1300 is generally curved much like the rear portion of the user's head. That is, the positioning and stabilizing structure 1300 is generally dome shaped.

In certain forms, a flexible and/or resilient material may be disposed around the rigid or semi-rigid material of the temporal connectors 1250. The flexible material may be more comfortable against the user's head, in order to improve wearability and provide soft contact with the user's face. In one form, the flexible material is a textile sleeve at is permanently or removably coupled to each temporal connector 1250.

In one form, a textile may be over-moulded onto at least one side of the rigidizer. In one form, the rigidizer may be formed separately to the resilient component and then a sock of user contacting material (e.g., Breath-O-Prene™) may be wrapped or slid over the rigidizer. In alternative forms, the user contacting material may be provided to the rigidizer by adhesive, ultrasonic welding, sewing, hook and loop material, and/or stud connectors.

In some forms, the user contacting material may be on both sides of the rigidizer, or alternatively may only be on the user contacting side (e.g., the user contacting side) of the rigidizer to reduce bulk and cost of materials.

In some forms, the temporal connectors 1250 are constructed from a flexible material (e.g., a textile), which may be comfortable against the user's skin, and may not require an added layer to increase comfort.

Some forms of the positioning and stabilizing structure 1300 may include only temporal connectors 1250. The temporal connectors 1250 may be shaped like temples or arms of eye-glasses, and may rest against the user's head in a similar manner. For example, the temporal arms 3304 may provide a force directed into lateral sides of the user's head (e.g., toward the respective temporal bone).

### 5.3.2.2 Posterior Support Portion

As shown in FIG. 5B, some forms of the positioning and stabilizing structure 1300 may include a rear support, e.g. a posterior support portion 1350 for assisting in supporting the display screen 1220 and/or the display unit housing 1205 proximate to the user's eyes. The posterior support portion 1350 may assist in anchoring the display screen 1220 and/or the display unit housing 1205 to the user's head in order to appropriately orient the display screen 1220 proximate to the user's eyes.

In some forms, the posterior support portion 1350 may be coupled to the display unit housing 1205 via the temporal connectors 1250.

In certain forms, the temporal connectors 1250 may be directly coupled to the display unit housing 1205 and to the posterior support portion 1350.

In some forms, the posterior support portion 1350 may have a three-dimensional contour curve to fit to the shape of a user's head. For example, the three-dimensional shape of the posterior support portion 1350 may have a generally round three-dimensional shape adapted to overlay a portion of the parietal bone and the occipital bone of the user's head, in use.

In some forms, the posterior support portion 1350 may be a posterior portion of the positioning and stabilizing structure 1300. The posterior support portion 1350 may provide an anchoring force directed at least partially in the anterior direction.

In certain forms, the posterior support portion 1350 is the inferior-most portion of the positioning and stabilizing structure 1300. For example, the posterior support portion 1350 may contact a region of the user's head between the occipital bone and the trapezius muscle. The posterior support portion 1350 may hook against an inferior edge of the occipital bone (e.g., the occiput). The posterior support portion 1350 may provide a force directed in the superior direction and/or the anterior direction in order to maintain contact with the user's occiput.

In certain forms, the posterior support portion 1350 is the inferior-most portion of the entire head-mounted display system 1000. For example, the posterior support portion 1350 may be positioned at the base of the user's neck (e.g., overlaying the occipital bone and the trapezius muscle more inferior than the user's eyes) so that the posterior support portion 1350 is more inferior than the display screen 1220 and/or the display unit housing 1205.

In some forms, the posterior support portion 1350 may include a padded material, which may contact the user's head (e.g., overlaying the region between the comfort to the user, and limit marks caused by the posterior support portion 1350 pulling against the user's head.

### 5.3.2.3 Forehead Support

As shown in FIGS. 5A and 5B, some forms of the positioning and stabilizing structure 1300 may include a forehead support 1360 that can contact the user's head superior to the user's eyes, while in use. For example, the forehead support 1360 may overlay the frontal bone of the user's head. In certain forms, the forehead support 1360 may also be more superior than the sphenoid bones and/or the temporal bones. This may also position the forehead support 1360 more superior than the user's eyebrows.

In some forms, the forehead support 1360 may be an anterior portion of the positioning and stabilizing structure 1300, and may be disposed more anterior on the user's head than any other portion of the positioning and stabilizing structure 1300. The posterior support portion 1350 may provide a force directed at least partially in the posterior direction.

In some forms, the forehead support 1360 may include a cushioning material (e.g., textile, foam, silicone, etc.) that may contact the user, and may help to limit marks caused by the straps of the positioning and stabilizing structure 1300. The forehead support 1360 and the interfacing structure 1100 may work together in order to provide comfort to the user.

In some forms, the forehead support 1360 may be separate from the display unit housing 1205, and may contact the user's head at a different location (e.g., more superior) than the display unit housing 1205.

In some forms, the forehead support 1360 can be adjusted to allow the positioning and stabilizing structure 1300 to accommodate the shape and/or configuration of a user's face.

In some forms, the temporal connectors 1250 may be coupled to the forehead support 1360 (e.g., on lateral sides of the forehead support 1360). The temporal connectors 1250 may extend at least partially in the inferior direction in order to couple to the posterior support portion 1350.

In certain forms, the positioning and stabilizing structure 1300 may include multiple pairs of temporal connectors 1250. For example, one pair of temporal connectors 1250 may be coupled to the forehead support 1360, and one pair of temporal connectors 1250 may be coupled to the display unit housing 1205.

In some forms, the forehead support 1360 can be presented at an angle which is generally parallel to the user's forehead to provide improved comfort to the user. For example, the forehead support 1360 may position the user in an orientation that overlays the frontal bone, and is substantially parallel to the coronal plane. Positioning the forehead support substantially parallel to the coronal plane can reduce the likelihood of pressure sores which may result from an uneven presentation.

In some forms, the forehead support 1360 may be offset from a rear support that contacts a posterior region of the user's head (e.g., an area overlaying the occipital bone and the trapezius muscle). In other words, an axis along a rear strap would not intersect the forehead support 1360, which may be disposed more inferior and anterior than the axis along the rear strap. The resulting offset between the forehead support 1360 and the rear strap may create moments that oppose the weight force of the display screen 1220 and/or the display unit housing 1205. A larger offset may create a larger moment, and therefore more assistance in maintaining a proper position of the display screen 1220 and/or the display unit housing 1205. The offset may be increased by moving the forehead support 1360 closer to the user's eyes (e.g., more anterior and inferior along the user's head), and/or increasing the angle of the rear strap so that it is more vertical.

### 5.3.2.4 Adjustable Straps

Portions of the positioning and stabilizing structure 1300 may be adjustable, in order to impart a selective tensile force on the display screen 1220 and/or the display unit housing 1205 in order to secure a position of the display screen 1220 and/or the display unit housing 1205.

In some forms, the display unit housing 1205 may include at least one loop or eyelet 1254, and at least one of the temporal connectors 1250 may be threaded through that loop, and doubled back on itself. The length of a strap of the positioning and stabilizing structure 1300 threaded through the respective eyelet 1254 may be selected by the user in order to adjust the tensile force. For example, threading a greater length through the eyelet 1254 may supply a greater tensile force.

In some forms, at least one of the temporal connectors 1250 may include an adjustment portion 1256 and a receiving portion 1258. The adjustment portion 1256 may be positioned through the eyelet 1254 on the display unit housing 1205, and may be coupled to the receiving portion 1258 (e.g., by doubling back on itself). The adjustment portion 1256 may include a hook material, and the receiving portion 1258 may include a loop material (or vice versa), so that the adjustment portion 1256 may be removably held in the desired position. In some examples, the hook material and the loop material may be Velcro.

In certain forms, the strap may be constructed at least partially from a flexible and/or resilient material, which may conform to a shape of the user's head and/or may allow the adjustment portion to be threaded through the eyelet 1254. For example, the adjustment portion(s) 1256 may be constructed from an elastic textile, which may provide an elastic, tensile force. The remained of the temporal connectors 1250 may be constructed from the rigid or semi-rigid material described above (although it is contemplated that additional sections of the temporal connectors 1250 may also be constructed from a flexible material).

### 5.3.2.4.1 Top Strap Portion

In some forms, the positioning and stabilizing structure 1300 may include a top strap portion, which may overlay a superior region of the user's head.

In some forms, the top strap portion may extend between an anterior portion of the head-mounted display system 1000 and a posterior region of the head-mounted display system 1000.

In some forms, the top strap portion may be constructed from a flexible material, and may be configured to compliment the shape of the user's head.

In certain forms, the top strap portion may be connected to the display unit housing 1205. For example, the top strap portion may be coupled to the superior face 1230. The top strap portion may also be coupled to the display unit housing 1205 proximate to a posterior end of the display unit housing 1205.

In certain forms, the top strap portion may be coupled to the forehead support 1360. For example, the top strap portion may be coupled to the forehead support 1360 proximate to a superior edge. The top strap portion may be connected to the display unit housing 1205 through the forehead support 1360.

In some forms, the top strap portion may be connected to the posterior support portion 1350. For example, the top strap portion may be connected proximate to a superior edge of the posterior support portion 1350.

In some forms, the top strap portion may overlay the frontal bone and the pariental bone of the user's head.

In certain forms, the top strap portion may extend along the sagittal plane as it extends between the anterior and posterior portions of the head-mounted display system 1000.

In certain forms, the top strap portion may apply a tensile force oriented at least partially in the superior direction, which may oppose the force of gravity.

In some forms, the top strap portion may be adjustable in order to impart a selective tensile force on the display screen 1220 and/or the display unit housing 1205 in order to secure a position of the display screen 1220 and/or the display unit housing 1205.

In certain forms, the display unit housing 1205 and/or the forehead support 1360 may include at least one loop or eyelet 1254, and the top strap portion may be threaded through that eyelet 1254, and doubled back on itself. The length of the top strap portion threaded through the eyelet 1254 may be selected by the user in order to adjust the tensile force provided by the positioning and stabilizing structure 1300. For example, threading a greater length of the top strap portion through the eyelet 1254 may supply a greater tensile force.

In some forms, the top strap portion may include an adjustment portion and a receiving portion. The adjustment portion may be positioned through the eyelet 1254, and may be coupled to the receiving portion (e.g., by doubling back on itself). The adjustment portion may include a hook material, and the receiving portion may include a loop material (or vice versa), so that the adjustment portion may be removably held in the desired position. In some examples, the hook material and the loop material may be Velcro.

5.4 Head-Mounted Display System with Forehead and/or Posterior Supports

FIGS. 12 and 13 show a head-mounted display system 1000, FIGS. 7A-11, 15 and 16A-16B show forehead supports 1360 and components thereof suitable for use in the head-mounted display system 1000, and FIGS. 14A-14C show posterior support pad assemblies 1370. The forehead supports 1360 and/or posterior support pad assemblies 1370 may form part of positioning and stabilising structures 1300 for a head-mounted display system 1000. The head-mounted display system 1000 and components thereof disclosed herein may be constructed and arranged in particular for use in AR or VR. It is to be understood that the positioning and stabilising structures 1300 and head-mounted display systems 1000 described herein, or individual features thereof, may be applied in any of VR, MR, AR or other artificial reality. The head-mounted display system 1000 may otherwise have any of the features, configurations, aspects, functions and the like as described elsewhere herein.

The head-mounted display system 1000 shown in FIGS. 12 and 13 may comprise a head-mounted display unit 1200 comprising a display unit housing 1205. The display unit housing 1205 may comprise a display. The display may as described elsewhere herein or as known in the art, in examples. In the example shown in FIG. 12, the head-mounted display unit 1200 is held in front of the user's face but without contacting the user's face below the user's eyes. In other examples, the head-mounted display unit 1200 may comprise an interfacing structure 1100 connected to the display unit housing 1205 and constructed and arranged to engage the user's face in use surrounding the user's eyes.

The positioning and stabilising structure 1300 shown in FIGS. 12 and 13 is constructed and arranged to hold the head-mounted display unit 1200 in an operable position on the user's head in use (e.g. such that the display is positioned in a viewing position anterior to the user's eyes and, in some particular examples, such that the interfacing structure 1100 engages the user's face to form a light seal, such as at least in forehead, sphenoid and cheek regions of the user's face).

In examples in which the head-mounted display unit 1200 comprises an interfacing structure 1100 which engages the user's face around a full periphery of the user's eyes, the interfacing structure 1100 may be configured to engage the sides of the user's face lateral of the user's eyes and engage the user's forehead in use. The interfacing structure 1100 may engage the user's cheeks, the sides of the user's face lateral of the user's eyes and the user's forehead. The interfacing structure 1100 may engage the user's face at regions overlying the user's nose, maxilla, zygomatic bones, sphenoid bones and frontal bones. The interfacing structure 1100 may engage the user's face in region shown in FIG. 4D, for example.

In the example shown in FIG. 12, the head-mounted display unit 1200 is supported in front of the user's face but does not contact the user's face. In such an example the head-mounted display unit 1200 may be sufficiently large in a width dimension to extend partially around the sides of the user's head to limit light leak from the sides of the user's eyes and may be sufficiently large in a height dimension to limit light leak from above and below the user's eyes. As will be described, in this example the head-mounted display system 1000 comprises a positioning and stabilising structure 1300 with a forehead support 1360 configured to engage the user's forehead.

### 5.4.1 Forehead Support

FIGS. 7A-7B and 8A-8B show a forehead support 1360. The forehead support 1360 may be incorporated into a head-mounted display system 1000 in the position of the forehead support 1360 shown in FIG. 12. The head-mounted display system 1000 comprises a positioning and stabilising structure 1300 constructed and arranged to hold a head-mounted display unit 1200 in an operable position on the user's head in use. The positioning and stabilising structure 1200 may comprise a forehead support 1360, such as those shown in FIGS. 7A-7B, 8A-8B, 15, 16A, 16B or in one of the variations disclosed herein.

In some examples of the present technology, the forehead support 1360 comprises a frame 1400 (which may also be identified as a forehead frame or anterior frame) attached to the head-mounted display unit 1200 in use. The forehead support 1360 may further comprise a resilient forehead pad 1410 attached to the frame 1400. The forehead pad 1410 may be configured to engage the user's forehead in use. In some examples, at least a portion of the frame 1400 may be able to flex to conform to the shape of the user's forehead in use. In particular examples, the frame 1400 may be configured to bias the forehead pad 1410 into a shape that is more curved than the user's forehead such that in use the forehead pad 1410 is conformed to the shape of the user's head. For example, when the forehead pad 1410 is moved into engagement with the user's forehead, it is splayed out to conform to the specific shape of the particular user's forehead.

As shown in FIGS. 7A and 7B in particular, the frame 1400 may comprise a central portion 1401, which may be structured to attach to the display unit housing 1205, and a pair of lateral portions 1402 having lateral ends able to move with respect to the central portion 1401 to conform to the shape of the user's forehead in use. The lateral portions 1402 may be identified as “wings”. The lateral ends may for example each be able to move at least partially medially and laterally. In some examples the lateral ends of the lateral portions 1402 may each be able to move at least partially anteriorly and posteriorly. The distance between the lateral ends of the lateral portions 1402 may be smaller than the width of the area of the user's forehead that the forehead support 1360 is intended to engage, such that when the forehead pad 1410 is brought into contact with the user's head, the lateral ends of the lateral portions 1402 are forced to move apart by a small amount when conforming to the user's forehead. The lateral portions 1402 being biased towards a shape that is more curved than the shape of the user's forehead may enable the forehead support 1360 to maintain good stable contact with the user's forehead in use. This feature may also advantageously allow the forehead support 1360 to apply pressure evenly across the user's forehead in use, for example as opposed to applying uncomfortably higher levels of pressure to the medial region of the user forehead in comparison to lateral regions.

In this example the frame 1400 comprises more than two portions able to flex to move with respect to each other, which may advantageously allow the frame 1400 to conform to a wide range of user head shapes and sizes comfortably and stably in use. FIG. 9 shows a frame 1400 of a forehead support 1360 according to one such example of the present technology. In this form each of the lateral portions 1402 comprises a superior lateral portion 1405 and an inferior lateral portion 1406 and the lateral ends 1402 comprise a superior lateral end of each superior lateral portion 1405 and an inferior lateral end of each inferior lateral portion 1406. In this example the superior lateral portions 1405 are able to move with respect to the inferior lateral portions 1406, which may allow the forehead support 1360 to conform to complex geometry and a wide range of forehead shapes and sizes. For example, each superior lateral portion 1405 may be able to flex independently of the respective inferior lateral portion 1406 (e.g. on the same side). The superior lateral ends may be able to move with respect to the inferior lateral ends.

Whether the lateral portions 1402 are formed from one, two or more independently moveable portions, in some examples, such at the example shown in FIG. 9, the frame 1400 may comprise a superior medial portion 1403 having a superior end able to move with respect to the central portion 1401 of the frame 1400 to conform to the shape of the user's forehead in use. For example, the superior end may be able to move at least partially anteriorly and posteriorly. Also shown in FIG. 9, the frame 1400 may comprise an inferior medial portion 1404 having an inferior end able to move with respect to the central portion 1401 of the frame 1400 to conform to the shape of the user's forehead in use. The inferior end for example may be able to move at least partially anteriorly and posteriorly. As shown in FIG. 9, the superior medial portion 1403 and inferior medial portion 1404 may both be medial of the lateral portions 1402.

In the example shown in FIGS. 7A-7B and 8A-8B, the central portion 1401 may comprise a connection portion 1407 positioned anteriorly with respect to the lateral portions 1402. The connection portion 140 may be configured to be attached to the display unit housing 1205 of the head-mounted display unit 1200, in some examples via an intermediate component.

### 5.4.2 Posterior Support Pad Assembly

In the example shown in FIGS. 12 and 13, the positioning and stabilising structure 1300 of the head-mounted display system 1000 comprises a posterior support portion 1350 configured to engage a posterior portion of the user's head in use, and a pair of lateral support portions 1330 connecting between the posterior support portion 1350 and the head-mounted display unit 1200 in use. The posterior support portion 1350 may be a portion of the positioning and stabilising structure 1300 positioned at the posterior of the user's head. With reference to FIGS. 14A and 14B, the posterior support portion 1350 may comprise a posterior support pad assembly 1370, which may comprise a posterior frame 1420 connected to the lateral support portions 1330 (optionally via an intermediate component), and a posterior pad 1421 being connected to the posterior frame 1420. The posterior pad 1421 is configured to contact the user's head in use.

At least a portion of the posterior frame 1420 may be able to flex to conform to the shape of a posterior surface of the user's head in use. For example, the posterior frame 1420 may be configured to bias the posterior pad 1421 into a shape that is more curved than the shape of the posterior surface of the user's head such that in use the posterior pad 1421 is conformed to the posterior surface of the user's head. The flexibility and bias in the posterior frame 1420 may produce a similar effect at the posterior of the user's head as the frame 1400 of the forehead support 1360 provides at the user's forehead. The posterior frame 1420 may comprise a central portion 1401 connected in use to the lateral support portions 1330 (optionally via an intermediate component).

With reference in particular to FIG. 14B, the posterior frame 1420 may comprise a pair of lateral portions 1402 having lateral ends able to move with respect to the central portion 1401 to conform to the shape of the posterior surface of the user's head in use. The lateral ends may for example each be able to move at least partially medially and laterally. In some examples the lateral ends of the lateral portions 1402 may each be able to move at least partially anteriorly and posteriorly. The distance between the lateral ends of the lateral portions 1402 may be smaller than the width of the area of the user's head that the posterior support pad assembly 1370 is intended to engage, such that when the posterior pad 1421 is brought into contact with the user's head, the lateral ends of the lateral portions 1402 are forced to move apart by a small amount when conforming to the posterior surface of the user's head. The lateral portions 1402 being biased towards a shape that is more curved than the shape of the posterior of the user's head may enable the posterior support pad assembly 1370 to maintain good stable contact with the posterior of the user's head in use. This feature may also advantageously allow the posterior support pad assembly 1370 to apply pressure evenly across the user's forehead in use, for example as opposed to applying uncomfortably higher levels of pressure to the medial region of the posterior surface of the user's head in comparison to lateral regions thereof.

In some examples, such as the example shown in FIGS. 14A and 14B, the posterior frame 1420 may comprise a superior medial portion 1403 having a superior end able to move with respect to the central portion 1401 of the posterior frame 1420 to conform to the shape of the posterior surface of the user's head in use. For example, the superior end may be able to move at least partially anteriorly and posteriorly. Also as shown in FIG. 14B, the posterior frame 1420 may comprise an inferior medial portion 1404 having an inferior end able to move with respect to the central portion 1401 of the posterior frame 1420 to conform to the shape of the posterior surface of the user's head in use. The inferior end for example may be able to move at least partially anteriorly and posteriorly. As shown in FIG. 14B, the superior medial portion 1403 and inferior medial portion 1404 may both be medial of the lateral portions 1402. The flexibility of the posterior frame 1420 and it being formed from multiple portions moveable independently of each other may advantageously provide the same benefits as these features of the forehead support 1360, such as the ability to conform well to a wide range of user head shapes and sizes and even distribution of pressure.

### 5.4.3 Forehead Pad Materials and Other Features

In the example illustrated in FIGS. 7A-7B and 8A-8B, the forehead pad 1410 comprises a foam layer. The foam layer may be relatively thick, for example 8 mm, 10 mm or within the range of 6-12 mm for example, and may provide for the forehead pad 1410 with resilience and enable it to conform to surface features of the user's forehead. The frame 1400 of the forehead support 1360 may provide for macroscopic adjustment of the shape of the forehead support 1360 while the forehead pad 1410 provides for microscopic adjustment of the shape of the user-contacting surface during conforming of the forehead support 1360 to the shape of the user's forehead. The forehead pad 1410 may comprise a textile user-contacting layer 1411, as shown in FIGS. 8A-8B. The textile-user contacting layer 1411 may be attached to a silicone backing layer. The textile layer 1411 may advantageously provide for a soft feel on the user's forehead. A silicone backing layer may provide a high level of durability to the user-contacting layer 1411 and may act as a barrier between the textile user-contacting layer 1411 and the foam layer, which may provide for good cleanability of the textile user-contacting layer 1411 and protect the foam layer from moisture and/or other substances. The textile user-contacting layer 1411 may be attached to and supported by a silicone peripheral portion 1416 connected to the frame 1400. The peripheral portion 1416 may be formed from silicone or other materials although the use of silicone may provide for good cleanability and it may bond well to the silicone backing layer of the textile user-contacting layer 1411.

The frame 1400 may be stiffer than the forehead pad 1410 and may be formed from a stiffer material than materials forming the forehead pad 1410, such as more rigid plastic material such as ABS, polycarbonate, nylon or the like but may be structured to be able to flex in the manner described above. Some portions may be structured so as to be substantially rigid.

FIG. 10A shows a forehead pad 1410 according to another example of the present technology. In this example the forehead pad 1410 comprise a silicone user contacting layer 1411. The silicone may be 0.75 mm thick, for example, or may be between 0.5 mm and 1 mm thick, or between 0.6 and 0.8 mm thick. The forehead pad 1410 may also comprise a foam layer as described above with reference to the FIG. 7A example. An advantage of a silicone user-contacting layer 1411 is that it may be higher cleanable/wipeable. The silicone user-contacting layer 1411 in this example comprises a plurality of channels 1414 formed therein configured to allow for transfer of moisture between the forehead pad 1410 and the user's forehead towards a periphery of the forehead pad 1410. In some examples, such as that shown in FIGS. 10A and 10B, the channels 1414 may be configured to allow for transfer of moisture towards lateral and/or superior edges of the forehead pad 1410. In the example shown in FIGS. 10A and 10B, the channels 1414 are oriented to extend substantially horizontally proximate the sagittal plane of the user's head in use and may be shaped to curve partially in a superior direction while extending laterally on each side of the sagittal plane. The channels 1414 may advantageously help draw sweat outwardly and upwardly by capillary action, which may for example help prevent sweat from migrating downwardly towards the user's eyes and/or help keep the user's forehead comfortably dry. The forehead pad 1414 may for example comprise between 5 and 30 channels, between 5 and 20 channels or between 10 and 15 channels. In some examples each channel comprises a diameter of between 0.2 and 0.6 mm, such as 0.4 mm for example.

The user-contacting layer 1411 of the forehead pad 1410 may be attached to the frame 1400 to encapsulate the inner foam layer. The user-contacting layer 1411 may be joined to the frame by a permanent connection, for example by one or more permanent snap clips which hold the periphery of the user-contacting layer 1411 to the frame 1400 on the anterior side of the frame. Alternatively, the user-contacting layer 1411 may be removably fitted to the frame. In some examples the foam layer may be glued to the frame 1400. The frame 1400 may comprise one or more openings on the anterior side thereof to allow for air escape during compression of the foam layer when the user dons the head-mounted display system 1000.

The posterior pad 1421 may be formed from any of the materials, layers structures and combinations thereof described as options for the forehead pad 1410.

In other examples the foam layer may be a layer of gel, silicone, spacer fabric or another suitable resilient cushioning material or structure.

### 5.4.4 Shaping Member

FIGS. 15, 16A and 16B show forehead supports 1360 according to further examples of the present technology. In each of these examples the forehead support 1360 comprises a shaping member 1430 attached the frame 1400. The shaping member 1430 is configured to impart a shape to the frame 1400. The shaping member 1430 may be more rigid than the frame and may be substantially rigid. As described above, the frame 1400 is able to flex and may have various portions able to flex with respect to each other. The shaping member 1430 may force the frame or portions thereof into a particular shape. The shaping member 1430 may be applied to any of the forehead supports 1360 or posterior support pad assemblies 1370 described herein.

In some examples, the shaping member 1430 is configured to attach to an anterior surface of the frame 1400, such as by a snap fit connection, press fit connection with a plurality of lugs, magnets or by an adhesive, for example. In the example shown in FIG. 15, the shaping member 1430 comprises a plate. In the example shown in FIG. 16A, the shaping member 1430 comprises a skeleton. FIG. 16B shows another shaping member 1430 in the form a ring-shaped skeleton. In other examples the shaping member 1430 may comprise a different form, such as a bar.

The shape imparted to the frame 1400 may correspond to the shape of the user's forehead. In some examples the shape imparted to the frame 1400 by the shaping member 1430 corresponds to one of a series of shapes or sizes, such as small, medium and large, or narrow and wide, or the like. In such examples a user may select the shaping member 1430 that provides the best fit for their forehead.

In some examples the shape imparted to the frame by the shaping member 1430 matches the shape of the user's forehead. That is, the shape matches the shape of a particular unique user. The user may scan their forehead and the shaping member 1430 may be formed based on the scan of the user's forehead. In some examples the user may scan their entire face and data representing the shape of the user's forehead may be extracted. In such examples the shaping member 1430 may be formed from 3D printing.

In some examples, the frame 1400 when shaped by the shaping member 1430 is configured to bias the forehead pad into a shape that is more curved than the user's forehead such that in use the forehead pad 1410 is conformed to the shape of the user's head.

The use of a shaping member 1430 may advantageously allow the user to customise the forehead support 1360 or posterior support pad assembly 1370, as the case may be, to provide a good fit to their head. A good fit may provide for a highly comfortable and/or stable head-mounted display system 1000 and may provide for a fit that remains comfortable for a longer duration of use of the head-mounted display system 1000.

FIG. 16B shows an example in which a frame 1400 similar to the frame 1400 shown in FIG. 9 is shaped with a shaping member 1430. In this example, the frame 1400 comprises a central portion 1401 structured to attach to the display unit housing 1205, and a pair of lateral portions 1402 having lateral ends able to move with respect to the central portion 1401. The shaping member 1430 in this example is constructed and arranged to conform the lateral portions 1402 to the shape of the user's forehead in use. Likewise, in the examples shown in FIGS. 15 and 16A, the shaping member 1430 conforms the lateral portions 1402.

In all of the examples shown in FIGS. 15, 16A and 16B, the lateral ends of the lateral portions 1402 are each able to be moved by the shaping member 1430 at least partially medially and laterally. The lateral ends may also each able to be moved by the shaping member 1430 at least partially anteriorly and posteriorly.

Referring to the example shown in FIG. 16B, each of the lateral portions 1402 comprises a superior lateral portion 1405 and an inferior lateral portion 1406 and the lateral ends comprise a superior lateral end of each superior lateral portion 1405 and an inferior lateral end of each inferior lateral portion 1406, each superior lateral portion 1405 being able to be flexed by the shaping member 1430 independently of the respective inferior lateral portion 1406.

The frame 1400 in this example also comprises a superior medial portion 1403 having a superior end able to move with respect to the central portion 1401 of the frame 1400 and the shaping member 1430 is constructed and arranged to conform the superior medial portion 1403 to the shape of the user's forehead in use. The superior end may be able to be moved by the shaping member 1430 at least partially anteriorly and posteriorly.

Similarly, the frame 1430 comprises an inferior medial portion 1404 having an inferior end able to move with respect to the central portion 1401 of the frame 1400 and the shaping member 1430 is constructed and arranged to conform the inferior medial portion 1404 to the shape of the user's forehead in use. The inferior end may be able to be moved by the shaping member 1430 at least partially anteriorly and posteriorly.

The central portion 1401 of the frame 1400 may comprise a connection portion 1407 positioned anteriorly with respect to the lateral portions 1402, the connection portion 1407 configured to be attached to the display unit housing 1205 of the head-mounted display unit 1200, as shown in FIGS. 15 and 16A for example.

In some examples of the present technology, the posterior support pad assembly 1370 comprises a shaping member 1430 attached to the posterior frame 1420, the shaping member 1430 configured to impart a shape to the posterior frame 1420. FIG. 14C shows one example.

Any of the features described above in relation to the shaping member 1430 provided to forehead supports 1360 are to be understood to be applicable to the shaping member 1430 when provided to a posterior support pad assembly 1370. For example, the shaping member may be more rigid than the posterior frame 1420. The shaping member 1430 may be configured to attach to a posterior surface of the posterior frame 1420, as shown in FIG. 14C for example. In this example the shaping member 1430 comprises a ring-shaped skeleton. The shape imparted to the posterior frame 1420 by the shaping member 1430 may correspond to the shape of a posterior surface of the user's head or may match the shape of the posterior surface of the user's head. In some examples the shaping member 1430 is formed based on a scan of the user's head.

In some examples, the posterior frame 1420 when shaped by the shaping member 1430 may be configured to bias the posterior pad 1421 into a shape that is more curved than the shape of the posterior surface of the user's head such that in use the posterior pad 1421 is conformed to the posterior surface of the user's head.

With reference to FIG. 14C, the posterior frame 1420 in this example comprises a pair of lateral portions 1402 having lateral ends able to move with respect to the central portion 1401, the shaping member 1430 being constructed and arranged to conform the lateral portions 1402 to the shape of the posterior surface of the user's head in use. The lateral ends are each able to be moved by the shaping member 1430 at least partially medially and laterally and/or at least partially anteriorly and posteriorly.

The posterior frame 1420 in this example further comprises a superior medial portion 1403 having a superior end able to move with respect to the central portion 1401 of the posterior frame 1420, the shaping member 1430 being constructed and arranged to conform the superior medial portion 1403 to the shape of posterior surface of the user's head in use. The superior end may be able to be moved by the shaping member 1430 at least partially anteriorly and posteriorly.

The posterior frame 1420 may comprise an inferior medial portion 1404, as shown in FIG. 14C, having an inferior end able to move with respect to the central portion 1401 of the posterior frame 1420, the shaping member 1430 being constructed and arranged to conform the inferior medial portion 1404 to the shape of the posterior surface of the user's head in use. The inferior end may be able to be moved by the shaping member 1430 at least partially anteriorly and posteriorly.

### 5.4.5 Concave Edge of Forehead Pad

Another feature of forehead supports 1360 disclosed herein is that the forehead pad 1410 may have a concave superior edge. With reference to the forehead pads 1410 shown in FIGS. 7A-7B, 8A-8B, 10A and 11, for example, the forehead pads 1410 comprise an inferior edge and a superior edge 1413, the superior edge 1413 having a superior midpoint M lying in the sagittal plane of the user's head in use. The superior edge 1413 in these examples is shaped to curve at least partially in a superior direction away from the superior midpoint M in use. That is, the superior edge 1413 may be concave, at least when viewed from an anterior direction while worn on the user's head in use. The inferior edge may comprise an inferior midpoint also lying in the sagittal plane of the user's head in use, and the inferior edge may be shaped to curve at least partially in the superior direction away from the inferior midpoint in use. That is, the inferior edge may be convex. The inferior edge may comprise a lesser curvature in the superior direction than the superior edge 1413. As shown in the drawings, the forehead pad 1410 may comprise a pair of rounded lateral sides connecting the superior and inferior edges, and may be substantially kidney-shaped.

This configuration, and in particular the superior edge curving towards a superior direction at lateral sides of the forehead pad 1410, may advantageously provide for a large contact area against the user's forehead without, or with a low risk of, impinging on the user's hair, which may be uncomfortable. The superior edge 1413 may follow the user's hairline. This may provide for even distribution of force over a large and wide area, which may reduce peak pressure and overall pressure, providing for good comfort and/or stability in use.

The posterior pad 1421 in the example shown in FIGS. 14A and 14B has a convex superior edge and convex inferior edge, which may advantageously provide for a large area of contact between the posterior pad 1421 and the user's head. The posterior pad 1421 may be substantially dome shaped.

### 5.4.6 Top Strap Portion

With reference to FIGS. 14A and 14B, in some examples of the present technology the head-mounted display system 1000 may comprise a top strap portion 1340 connecting between a posterior support portion 1350 and a forehead support 1360 in use. The top strap portion 1340 may comprise a pair of anterior portions 1341 and a posterior portion 1342, the anterior portions 1341 connecting to the forehead support 1360 at respective lateral sides of the forehead support 1360. The top strap portion 1340 may be generally Y-shaped as shown by the combination of FIGS. 14A and 14B and a junction between the anterior portions 1341 and the posterior portion 1342 may lie in a coronal plane aligned with or proximate to each otobasion superior of the user's head. The posterior portion 1342 may lie in the sagittal plane in use and the anterior portions 1341 may extend from the posterior portion 1342 anteriorly and partially laterally to connect to the forehead support 1362.

The anterior portions 1341 of the top strap portion 1340 may be connected to the frame 1400 of the forehead support 1360. In the case of the forehead support 1360 shown in FIG. 7A, the anterior portions 1341 of the top strap portion 1340 may connect to superior portions of the frame 1400 as depicted in FIG. 12, for example. However, in another example, shown in FIG. 11, the frame 1400 may comprise a pair of superior extension portions 1415 extending superiorly proximate lateral corners of the frame 1400. The anterior portions 1341 of the top strap portion 1340 may be connected to respective superior extension portions 1415. For example, anterior ends of the of the anterior portions of the top strap portion 1340 may connect to attachment points 1417, which may be located at the ends of the superior extension portions 1415 as shown in FIG. 11. The connection may be a snap fit, buckle, magnet or other suitable connection, for example. The superior extension portions 1415 may be configured to extend superiorly and partially posteriorly and/or partially medially. The partially posterior and/or medial extension of the superior extension portions 1415 may help urge the lateral and superior corners of the forehead pad 1410 into the user's forehead, which may help provide even pressure and/or good stability in use.

The posterior portion 1342 of the top strap portion 1340 may be connected to the posterior support portion 1350 of the positioning and stabilising structure 1300. In examples in which the posterior support portion 1350 comprises a posterior support pad assembly 1370, the posterior portion 1342 of the top strap portion 1340 may be connected to the posterior support pad assembly 1370. In some particular examples, the posterior portion of the top strap portion 1340 may be connected to a posterior frame 1420 of the posterior support pad assembly 1370. The posterior portion 1342 of the top strap portion 1340 may be adjustably connected. For example, the posterior portion 1342 may pass through an opening in the posterior frame 1400, or other portion to which it is connected, and may be looped back and secured to itself, for example with a hook-and-loop connection, clip or other suitable connection. Advantageously, the posterior portion 1342 of the top strap portion 1340 being adjustable as opposed to the anterior portions 1341 provides the user with a single adjustment point, which may make the head-mounted display system 1000 easier to adjust and more user friendly.

In other examples, the positioning and stabilising structure 1300 may comprise only a single strap portion forming the top strap portion 1340, for example a single strap aligned with the sagittal plane of the user's head.

Another adjustment mechanism in the FIGS. 14A and 14B example is that the positioning and stabilising structure 1300 comprises a dial adjustment mechanism 1390 comprising a rotatable dial, the dial adjustment mechanism 1390 being configured to cause a change in length of the lateral support portions 1330 when the dial is rotated. The dial adjustment mechanism 1390 may be provided to the posterior support portion 1350, for example, and may be positioned in the sagittal plane of the user's head in use.

### 5.5 Self-Tensioning and Releasing Headgear

FIG. 17A shows a head-mounted display system 1000 according to another example of the present technology. FIG. 17B shows a schematic illustration of a portion thereof. The head-mounted display system 1000 shown in FIG. 17A and the positioning and stabilising structure 1300 thereof may be constructed and arranged in particular for use in VR. However, it is to be understood that the positioning and stabilising structures 1300 and head-mounted display systems 1000 described herein, or individual features thereof, may be applied in any of VR, MR, AR or other artificial reality. The head-mounted display system 1000 may otherwise have any of the features, configurations, aspects, functions and the like as described elsewhere herein.

The head-mounted display system 1000 shown in FIG. 17A may comprise a head-mounted display unit 1200 comprising a display unit housing 1205. The display unit housing 1205 may comprise a display. The display may be as described elsewhere herein or as known in the art. The head-mounted display unit 1200 may comprise an interfacing structure 1100 connected to the display unit housing 1205 and constructed and arranged to engage the user's face in use.

The positioning and stabilising structure 1300 shown in FIGS. 17A-17D is constructed and arranged to hold the head-mounted display unit 1200 in an operable position on the user's head in use (e.g., such that the display is positioned in a viewing position anterior to the user's eyes and such that the interfacing structure 1100 engages the user's face to form a light seal, such as at least in forehead, sphenoid and cheek regions of the user's face).

### 5.5.1 Interfacing Structure

The interfacing structure 1100 may be configured to engage the user's face around a periphery of the user's eyes in use. The interfacing structure 1100 may be configured to engage the sides of the user's face lateral of the user's eyes and engage the user's forehead in use. The interfacing structure 1100 may engage the user's cheeks, the sides of the user's face lateral of the user's eyes and the user's forehead. The interfacing structure 1100 may engage the user's face at regions overlying the user's nose, maxilla, zygomatic bones, sphenoid bones and frontal bones. The interfacing structure 1100 may engage the user's face in regions shown in FIG. 4D, for example.

The interfacing structure 1100 may comprise a face engaging flange structured and arranged to be provided around a periphery of an eye region of the user's face and configured to engage the user's face in use. The face engaging flange may be flexible and resilient and may be in the form of a membrane. The face engaging flange may be formed from an elastomeric material, for example silicone or TPE. In some examples the interfacing structure 1100 comprises a cushion (not visible in the drawings). The cushion may be at least partially covered by the face engaging flange. The cushion may be formed from foam, silicone, gel or another suitable cushion material or structure constructed to behave as a cushion. In some examples, the interfacing structure 1100 comprises a cushion but no face engaging flange.

The interfacing structure 1100 may be configured to engage the user's face and resist compression when the head-mounted display unit 1200 is fastened securely to the user's face, while remaining comfortable to the user. The cushion (if present) may contribute to the resilience of the interfacing structure 1100. The interfacing structure 1100 may comprise a pair of cheek portions configured to engage the user's cheeks in use, a forehead portion configured to engage the user's forehead in use, and a pair of sphenoid portions located on respective lateral sides of the interfacing structure connecting between the forehead portion and the cheek portions and configured to engage the user's head proximate the sphenoid bone.

In some examples, the interfacing structure 1100 comprises a nasal portion between the cheek portions. The nasal portion may be configured to engage the user's nose in use and may be configured to at least partially block light from reaching the user's eyes from the user's nose region (e.g., block light travelling via a path proximate the surfaces of the user's nose). The nasal portion may for example be configured to engage anterior, superior and/or lateral surfaces of the user's nose in use. The nasal portion may be attached to the cheek portions.

### 5.5.2 Arms

The positioning and stabilising structure 1300 in the example shown in FIGS. 17A-17D comprises a pair of arms 1210 connected in use (e.g., fixedly attached or removably connected) to respective lateral sides of the display unit housing 1205. The arms 1210 are in this example semi-rigid and are constructed and arranged to project at least partially posteriorly from the display unit housing 1205 on respective lateral sides of the user's head in use. Each of the arms 1210 may include a connector (e.g., snap-fit connector) to facilitate removeable connection to the display unit housing 1205.

The arms 1210 may be configured to resist bending about one or more axes and may therefore be described as rigidised or at least semi-rigid. In some examples the arms 1210 may be substantially rigid. The arms 1210 may comprise a textile outer layer and may be rigidised to be substantially rigid. In some examples the arms 1210 may comprise a substantially rigid component covered in a textile layer (e.g., textile on both sides or textile on one side). In some examples only a user-facing side of the arms comprises a textile layer. In some examples the arms 1210 may be constructed to resist bending towards superior and inferior directions but allow for bending medially and laterally, to accommodate a range of head shapes and sizes.

In some examples each arm 1210 is at least partially covered by a textile sleeve. In some particular examples each arm 1210 is encapsulated within a textile sleeve. Partially or fully covering the arms 1210 may provide for comfortable contact between the arm 1210 and the user's head, may be aesthetically pleasing, may provide for a perception of comfort and may conceal or protect other components provided to the arms 1210, such as electronics, sensors, transducers or the like, or wiring running along an arm 1210 to a posterior battery pack or any other components.

### 5.5.3 Band Strap Portion

The positioning and stabilising structure 1300 of the head-mounted display system 1000 shown in FIG. 17A comprises a band strap portion 1380 constructed and arranged to encircle the user's head in use to engage a region of the user's head overlying the frontal bone and engage a posterior region of the user's head. In some examples the band strap portion 1380 forms an occipital strap portion 1320 constructed and arranged to engage the posterior region of the user's head, the posterior region of the user's head overlying or lying inferior to the occipital bone. In other examples the posterior region of the user's head engaged by the band strap portion 1380 may overlie the parietal bones of the user's head. The band strap portion 1380 may fit snugly around the user's head in use. The band strap portion 1380 engages the user's head and provides an anchor to which other portions of the head-mounted display system 1000 or positioning and stabilising structure 1300 thereof connect.

In the example shown in FIGS. 17A-17D, the positioning and stabilising structure 1300 also comprises a parietal strap portion 1310 which together with the band strap portion 1380 secures the head-mounted display system 1000 on the user's head. In other examples the positioning and stabilising structure 1300 may not comprise a parietal strap portion 1310 and instead the band strap portion 1380 may engage the user's head sufficiently snugly that it is able to hold the head-mounted display system 1000 on the user's head alone. The parietal strap portion 1310 may extend between lateral portions of the band strap portion 1380, for example between locations overlying temporal bones of the user's head. The parietal strap portion 1310 may be substantially inextensible or may have a low extensibility to allow a snug fit while providing support. The parietal strap portion 1310 may be formed from a textile material, for example a tubular knitted textile material. The spaced relationship between the parietal strap portion 1310 and the occipital strap portion 1320 may help stabilize the positioning and stabilising structure, as described earlier.

The band strap portion 1380 is configured to be in tension in use. The tension in the band strap portion 1380 ensures it engages the user's head snugly and therefore helps to hold the positioning and stabilising structure 1300 on the user's head in use.

In the example shown in FIG. 17A, the band strap portion 1380 comprises an anterior portion 1380a of the band strap portion 1380 and a posterior portion 1380b of the band strap portion 1380. The posterior portion 1380b may alternatively be identified as an inferior portion in examples in which it is more inferior than the anterior portion 1380a. Likewise the anterior portion 1380a may alternatively be identified as a superior portion in examples in which it is more superior than the posterior portion 1380b. The posterior portion 1380b also forms the occipital strap portion 1320 of the positioning and stabilising structure 1300 in this example.

The band strap portion 1380 may comprise a textile outer layer, for example in the form of a textile sleeve, which may be knitted. The band strap portion 1380 may also comprise, or function as, a rigidiser. For example, the band strap portion 1380 may comprise a rigidiser layer, or a rigidiser member configured to resist deformation of the band strap portion 1380. The rigidiser layer or member may be formed from a thermoplastic elastomer, such as Hytrel, in some examples. The rigidiser may be inside of a textile sleeve. In one example, the band strap portion 1380 comprises a textile sleeved Hytrel rigidiser. The band strap portion 1380 may be partially flexible to conform to the shape of the user's head. All of these options are to be understood as options applicable to the anterior portion 1380a of the band strap portion 1380, the posterior portion 1380b of the band strap portion 1380, or both. The parietal strap portion 1310, if included, may may be formed from a textile material and may be elastic. The parietal strap portion 1310 may be attached to the band strap portion 1380 by sewing, welding (e.g., ultrasonic welding), gluing or by another suitable manner of connection.

In other examples of the present technology, some or all of the portions of the positioning and stabilising structure 1300 may comprise materials other than or in addition to textile or textile and foam. In some examples some or all strap portions may comprise silicone, rigid plastic, foam, leather or another suitable material.

The band strap portion 1380 may be adjustable in length and may comprise a dial adjustment mechanism comprising a rotatable dial. The dial adjustment mechanism may be configured to cause a change in length of the band strap portion 1380 when the dial is rotated. The dial adjustment mechanism may be provided, for example, in the occipital strap portion 1320 of the band strap portion 1380. The dial may be positioned proximate the mid-sagittal plane (e.g., intersecting the sagittal plane) of the user's head in use. In examples in which the positioning and stabilising structure 1300 comprises a dial adjustment mechanism, the occipital strap portion 1320/inferior strap portion 1380b may be formed in two portions, a left portion and a right portion, connected by the dial adjustment mechanism so as to allow an amount of overlap between the left portion and the right portion to be adjusted when the user rotates the dial of the dial adjustment mechanism.

### 5.5.3.1 Lateral Connections

In the example shown in FIGS. 17A and 17B, the head-mounted display unit 1200 is attached to the band strap portion 1380 at a pair of lateral connections 1301. In the FIG. 17A example, the pair of arms 1210 each connect to the band strap portion 1380 at a respective lateral connection 1301. Each lateral connection 1301 may be positioned superior to the user's otobasion superior and may be aligned proximate a mid-coronal plane aligned with each otobasion superior of the user. Each arm 1210 may be pivotably attached to one or both of the anterior portion 1380a and posterior portion 1380b of the band strap portion 1380.

In this example the head-mounted display unit 1200 is able to be lifted upwardly by the user away from a viewing position to remove the head-mounted display system 1000 from the user's head. This lifting upward motion may include a motion in which the user pivots the head-mounted display unit 1200 upwardly substantially about an axis through the two lateral connections 1310. The lifting upward motion may then involve the user continuing to lift the head-mounted display unit 1200 superiorly and/or posteriorly to remove/doff the head-mounted display system 1000 from the head entirely. In the example shown in FIG. 17A, when the head-mounted display unit 1200 is lifted upwardly away from the viewing position, the head-mounted display unit 1200 rotates at the lateral connections 1301, which in this example relieves tension in the band strap portion 1380. This relief/reduction of tension in band strap portion 1380 facilitates removal of the head-mounted display system 1000 from the user's head as the band strap portion 1380, when under tension, functions to hold the head-mounted display system 1000 in position on the user's head. Removing that tension allows the head-mounted display system 1000 to be pulled upwardly and/or rearwardly off the user's head.

In reverse, when the user puts the head-mounted display system 1000 on their head, lowering the head-mounted display unit 1200 into the viewing position, after the positioning and stabilising structure 1300 is placed over the top and rear of the user's head, the lowering and downwardly pivoting movement of the head-mounted display unit 1200 about the lateral connections 1301 results in tensioning of the band strap portion 1380, which provides a snug fit to securely and comfortably hold the head-mounted display system 1000 on the user's head.

As shown in FIGS. 17B, 17C and 17D and noted above, in this example the band strap portion 1380 is formed from an anterior portion 1380a of the band strap portion 1380 and a posterior portion 1380b of the band strap portion 1380. The anterior portion 1380a and the posterior portion 1380b each have corresponding ends at the respective lateral connections 1301. At each lateral connection 1301 the head-mounted display unit 1200 connects to respective ends of the anterior portion 1380a and the posterior portion 1380b. When the head-mounted display unit 1200 is in the viewing position (FIGS. 17A-17C), at each lateral connection 1301 the respective ends of the anterior portion 1380a and the posterior portion 1380b of the band strap portion 1380 are pulled towards each other. This tensions the band strap portion 1380 by pulling ends of the two portions forming the band strap portion 1380 together, facilitating a snug fit to the user's head. However, when the head-mounted display unit 1200 is lifted upwardly away from the viewing position (FIG. 17D), at each lateral connection the respective ends of the anterior portion 1380a and the posterior portion 1380b of the band strap portion 1380 are allowed to move apart. As the ends of the two portions of the band strap portion 1380 are allowed to move apart, tension in the band strap portion 1380 as a whole is relieved, allowing the positioning and stabilising structure 1300 to slip off the user's head or at least be more easily pulled off than otherwise.

As shown in FIGS. 17B-17D, the head-mounted display unit 1200 connects to the anterior portion 1380a and posterior portion 1380b of the band strap portion 1380 at connection points 1302. The connections points 1302 may be between the arms 1210 and the band strap portion 1380. The connection points 1302 are arranged such that upon rotation of the connection points 1302, the tension in the band strap portion 1380 is varied.

FIGS. 17C and 17D show the positioning and stabilising structure 1300 in the viewing position and release position, respectively. In the viewing position of the head-mounted display unit 1200, shown in FIG. 17C, a central axis C of each arm 1210 is aligned anteriorly (in some examples the central axis C may be aligned partially inferiorly as shown in FIG. 17C, and in other examples may be aligned partially superiorly). When the head-mounted display unit 1200 is lifted, or during removal, as shown in FIG. 17D, the central axis C of each arm 1210 is aligned more superiorly than in the viewing position, for example partially anteriorly and partially superiorly. In the viewing position the central axis C of each arm 1210 may be aligned towards the user's eyes while during release the central axis C of each arm 1210 may be aligned towards the user's forehead, or higher on the user's forehead than when in the viewing position.

When the head-mounted display unit 1200 is lifted upwardly it rotates at the lateral connections 1301, which causes the connection points 1302 to rotate such that they are spaced further apart along the length of the band strap portion 1380. When in the viewing position as shown in FIG. 17C, the connection points 1302 are close together along the length of the band strap portion 1380 and the two portions of the band strap portion 1380 (the anterior portion 1380a and posterior portion 1380b) are spaced by a small spacing S₁. In some examples the spacing S₁ may be zero or even negative (e.g. there may be an overlap between the ends of the anterior portion 1380a and posterior portion 1380b). During release, in the lifted position shown in FIG. 17D, the connection points 1302 move further apart along the length of the band strap portion 1380 and the two portions of the band strap portion 1380 (the anterior portion 1380a and posterior portion 1380b) are spaced by a spacing S₂ that is larger than the spacing S₁. The connection points 1302 may be forced to moved by the arms 1210 during lifting/pivoting of the head-mounted display unit 1200 although may also be urged apart by tension in the band strap portion 1380.

As shown in FIGS. 17B-17D and explained above, each arm 1210 connects to the anterior portion 1380a and posterior portion 1380b of the band strap portion 1380 at respective connection points 1302 between said arm 1210 and the anterior portion 1380a and posterior portion 1380b of the band strap portion 1380. Each connection point 1302 may be a rotatable connection. For example, each connection point 1302 may be a pin connection. Pins may be provided to the arms 1210 and holes to rotatably receive the pins, for example rigid eyelets, may be provided to the band strap portion 1380. Also as shown in FIGS. 17C and 17D, the connection points 1302 on the anterior portion 1380a of the band strap portion 1380 are positioned proximate an inferior edge of the anterior portion 1380a of the band strap portion 1380. The connection points 1302 on the posterior portion 1380b of the band strap portion 1380 are positioned proximate a superior edge of the posterior portion 1380b of the band strap portion 1380. As illustrated in FIGS. 17C and 17D, the connection points 1302 being positioned in this manner allows the connection points to be closer together along the length the band strap portion 1380 when the head-mounted display unit 1200 is in the viewing position shown in FIG. 17C than when the head-mounted display unit 1200 is in a lifted position as shown in FIG. 17D. In particular, the connection points 1302 are aligned, in the viewing position shown in FIG. 17C, at a larger angle with respect to the path along which the band strap portion 1380 lies, than they are in the lifted position shown in FIG. 17D.

FIGS. 18A, 18B and 19 show positioning and stabilising structures 1300 according to further examples of the present technology. In these examples, each positioning and stabilising structure 1300 comprises a cam mechanism 1385 that tensions the band strap portion 1380 when the head-mounted display unit 1200 is moved into the viewing position and releases tension in the band strap portion 1380 when the head-mounted display unit 1200 is lifted upwardly away from the viewing position. The positioning and stabilising structure 1300 in these examples comprises a cam mechanism on each lateral side of the positioning and stabilising structure 1300. In other examples there may be a single cam mechanism provided on one lateral side, configured to tension the entire band strap portion 1380.

In some examples, such as those shown in FIGS. 18A, 18B and 19, the cam mechanism 1385 comprises a cam 1381 and one of the pair of arms 1210 rotates the cam 1381 during movement of the head-mounted display unit 1200 towards or away from the viewing position to operate the cam mechanism 1385. The cam 1381 may be connected to or formed integrally with the arm 1210 or may be engaged by the arm 1210, in various examples.

In some examples the cam mechanism 1385 or the cam 1381 thereof is constructed and arranged to resist movement of the head-mounted display unit 1200 away from the viewing position when the head-mounted display unit 1200 is in the viewing position. This may make the viewing position a stable position in comparison to an arrangement in which the tension in the band strap portion 1380 is continuously urging the head-mounted display unit 1200 to move away from the viewing position. The cam mechanism 1385 may resist movement of the head-mounted display unit 1200 away from the viewing position by generating an initial increase in tension in the band strap portion 1380 when the user lifts the head-mounted display unit 1200 upwardly away from the viewing position. After the initial increase in tension the cam mechanism 1385 relieves tension in the band strap portion 1380 to allow doffing of the head-mounted display system 1000.

The cam mechanism 1385 may operate to impart or allow relative movement of the respective ends of the anterior portion 1380a and posterior portion 1380b of the band strap portion 1380. In some examples, the cam mechanism 1385 comprises a cam 1381 and a tether 1382 connecting one of the anterior portion 1380a of the band strap portion 1380 and the posterior portion 1380b of the band strap portion 1380 to the cam 1381. Rotation of the cam 1381 during movement of the head-mounted display unit 1200 towards the viewing position pulls said respective one of the anterior portion 1380a or posterior portion 1380b towards the cam 1381 to increase tension in the band strap portion 1380.

In the example shown in FIGS. 18A and 18B, the cam mechanism 1385 comprises a cam 1381 connected to each arm 1210 of the positioning and stabilising structure 1300. The cam 1381 may be in the form of a wheel but may not be circular. The anterior portion 1380a of the band strap portion 1380 is in this example rotatably connected to the arm 1210 and/or cam 1381. A tether 1382 connects the posterior portion 1380b of the band strap portion 1380 to the cam 1381. As shown in FIG. 18A, the arm 1210 is in a position corresponding to a viewing position of the head-mounted display unit 1200 in use and the cam 1381 is rotated to draw the end of the posterior portion 1380b of the band strap portion 1380 via the tether 1382 towards the anterior portion 1380a, generating tension in the band strap portion 1380. As shown in FIG. 18B, the arm 120 is in a position corresponding to a lifted position of the head-mounted display unit 1200, such as during doffing/removal of the head-mounted display system 1000, and the cam 1381 is rotated clockwise in comparison to its position in FIG. 18A, which releases tension the band strap portion 1380. As shown by comparison of the two positions of the arms 1210 in FIGS. 18A and 18B, the spacing S₁ between the respective ends of the anterior portion 1380a and 1380b in FIG. 18A is less than the spacing S₂ between the respective ends in FIG. 18B.

FIG. 19 shows another example of the positioning and stabilising structure 1300 comprise a cam mechanism 1385. In this example the cam mechanism 1385 comprises a cam 1381 and a tether 1382 connecting the anterior portion 1380a of the band strap portion 1380 to the posterior portion 1380b of the band strap portion 1380. In this example, the cam 1381 constructed and arranged to engage the tether 1382 to draw the anterior portion 1380a and posterior portion 1380b of the band strap portion 1380 together during movement of the head-mounted display unit 1200 towards the viewing position. The cam is in the form of an arm or lever, in this example.

In some examples, the tether is constructed from an elastically extendable material. In other examples, the tether is non-elastic. In some example, the tether is non-elastic but the band strap portion 1380, or a portion thereof, such as one or both of the anterior portion 1380a and posterior portion 1380b, is elastically extendable. A portion of the band strap portion 1380, or tether 1382, being elastically extendable may facilitate a snug, comfortable and stable fit of the band strap portion 1380 to the user's head in use.

### 5.6 Cleaning

In some forms, the head-mounted display system 1000 or at least a portion thereof, is designed to be used by a single user, and cleaned in a home of the user, e.g., washed in soapy water, without requiring specialised equipment for disinfection and sterilisation. Specifically, the positioning and stabilizing structure 1300 and the interfacing structure 1100 are designed to be cleaned, as they are both in direct contact with the user's head.

In some other forms, the components of the positioning and stabilizing structure 1300 and interfacing structure 1100 are used in labs, clinics and hospitals wherein a single head-mounted display may be reused on multiple persons or used during medical procedures. In each of the labs, clinics and hospitals the head-mounted displays, or relevant components thereof, can be reprocessed and be exposed to, for example, processes of thermal disinfection, chemical disinfection and sterilisation. As such, the design of the positioning and stabilizing structure and interfacing structure may need to be validated for disinfection and sterilisation of the mask in accordance with ISO17664.

Materials may be chosen to withstand reprocessing. For example, robust materials may be used in the positioning and stabilizing structure 1300 to withstand exposure to high level disinfection solutions and agitation with a brush. Further, some components of the positioning and stabilizing structure are separable, and in-use may be disconnected to improve the reprocessing efficacy.

In some examples, the interfacing structure 1100 may, in use, be in contact with the user's head and therefor may become dirty (e.g., from sweat). The interfacing structure 1100 may be designed to be removed from the display unit housing 1205, to provide the ability to remove it for cleaning and/or replacement. It may be desirable to wash the interfacing structure 1100 while not getting the positioning and stabilizing structure 1300 wet. Alternatively or in addition, the positioning and stabilizing structure 1300 may be dirty from contact with the user's head, and may be removed for cleaning and/or replacement independently of the interfacing structure 1100. In either case, this may be facilitated by allowing these components to disconnect for such a purpose.

In some examples, a cover (e.g., constructed from a textile, silicone, etc.) may be removably positioned over the interfacing structure and can be removed to be cleaned and/or replaced after each use. The cover may allow the interface structure 3400 to remain fixed to the display unit housing 1205, and still provide a surface that can be easily cleaned after being used.

### 5.7 External Computer

In some forms, the head-mounted display system 1000 (e.g., VR, AR, and/or MR) may be used in conjunction with a separate device, like a computer or video game console. For example, the display interface may be electrically connected to the separate device.

In some forms, at least some processing for the head-mounted display system 1000 may be performed by the separate device. The separate device may include a larger and/or more powerful processor than could be comfortably supported by the user (e.g., the processor of the separate device may be too heavy for the user to comfortably support on their head).

### 6 GLOSSARY

For the purposes of the present technology disclosure, in certain forms of the present technology, one or more of the following definitions may apply. In other forms of the present technology, alternative definitions may apply.

### 6.1 General

Ambient: In certain forms of the present technology, the term ambient will be taken to mean (i) external of the display interface and/or user, and (ii) immediately surrounding the display interface and/or user.

For example, ambient light with respect to a display interface may be the light immediately surrounding the user, e.g. the light in the same and/or adjacent room as a user, and/or natural light from the sun.

In certain forms, ambient (e.g., acoustic) noise may be considered to be the background noise level in the room where a user is located, other than for example, noise generated by the display device or emanating from speakers connected to the display device. Ambient noise may be generated by sources outside the room.

Leak: The word leak will be taken to be an unintended exposure to light. In one example, leak may occur as the result of an incomplete seal between a display unit and a users' face.

Noise, radiated (acoustic): Radiated noise in the present document refers to noise which is carried to the user by the ambient air. In one form, radiated noise may be quantified by measuring sound power/pressure levels of the object in question according to ISO 3744.

User: A person operating the display interface and/or viewing images provided by the display interface. For example, the person may be wearing, donning, and/or doffing the display interface.

### 6.1.1 Materials

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240

Polycarbonate: a thermoplastic polymer of Bisphenol-A Carbonate.

### 6.1.2 Mechanical Properties

Resilience: Ability of a material to absorb energy when deformed elastically and to release the energy upon unloading.

Resilient: Will release substantially all of the energy when unloaded. Includes e.g. certain silicones, and thermoplastic elastomers.

Hardness: The ability of a material per se to resist deformation (e.g. described by a Young's Modulus, or an indentation hardness scale measured on a standardised sample size).
‘Soft’ materials may include silicone or thermo-plastic elastomer (TPE), and may, e.g. readily deform under finger pressure.
‘Hard’ materials may include polycarbonate, polypropylene, steel or aluminium, and may not e.g. readily deform under finger pressure.

Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

Floppy structure or component: A structure or component that will change shape, e.g. bend, when caused to support its own weight, within a relatively short period of time such as 1 second.

Rigid structure or component: A structure or component that will not substantially change shape when subject to the loads typically encountered in use. An example of such a use may be setting up and maintaining a user interface in sealing relationship.

As an example, an I-beam may comprise a different bending stiffness (resistance to a bending load) in a first direction in comparison to a second, orthogonal direction. In another example, a structure or component may be floppy in a first direction and rigid in a second direction.

### 6.2 Materials

Closed-cell foam: Foam comprising cells that are completely encapsulated, i.e. closed cells.

Elastane: A polymer made from polyurethane.

Elastomer: A polymer that displays elastic properties. For example, silicone elastomer.

Ethylene-vinyl acetate (EVA): A copolymer of ethylene and vinyl acetate.

Fiber: A filament (mono or poly), a strand, a yarn, a thread or twine that is significantly longer than it is wide. A fiber may include animal-based material such as wool or silk, plant-based material such as linen and cotton, and synthetic material such as polyester and rayon. A fiber may specifically refer to a material that can be interwoven and/or interlaced (e.g., in a network) with other fibers of the same or different material.

Foam: Any material, for example polyurethane, having gas bubbles introduced during manufacture to produce a lightweight cellular form.

Neoprene: A synthetic rubber that is produced by polymerization of chloroprene. Neoprene is used in trade products: Breath-O-Prene.

Nylon: A synthetic polyamide that has elastic properties and can be used, for example, to form fibres/filaments for use in textiles.

Open-cell foam: Foam comprising cells, i.e. gas bubbles that aren't completely encapsulated, i.e. open cells.

Polycarbonate: a typically transparent thermoplastic polymer of Bisphenol-A Carbonate.

Polyethylene: A thermoplastic that is resistant to chemicals and moisture.

Polyurethane (PU): A plastic material made by copolymerizing an isocyanate and a polyhydric alcohol and, for example, can take the form of foam (polyurethane foam) and rubber (polyurethane rubber).

Semi-open foam: Foam comprising a combination of closed and open (encapsulated) cells.

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240.

Spacer Fabric: A composite construction comprised of two outer textile substrates joined together and kept apart by an intermediate layer of monofilaments.

Spandex: An elastic fibre or fabric, primarily comprised of polyurethane. Spandex is used in trade products: Lycra.

Textile: A material including at least one natural or artificial fiber. In this specification, a textile may refer to any material that is formed as a network of interwoven and/or interlaced fibers. A type of textile may include a fabric, which is constructed by interlacing the fibers using specific techniques. These include weaving, knitting, crocheting, knotting, tatting, tufting, or braiding. Cloth may be used synonymously with fabric, although may specifically refer to a processed piece of fabric. Other types of textiles may be constructed using bonding (chemical, mechanical, heat, etc.), felting, or other nonwoven processes. Textiles created through one of these processes are fabric-like, and may be considered synonymous with fabric for the purposes of this application.

Thermoplastic Elastomer (TPE): Are generally low modulus, flexible materials that can be stretched at room temperature with an ability to return to their approximate original length when stress is released. Trade products that use TPE include: Hytrel, Dynaflex, Medalist

Thermoplastic Polyurethane (TPU): A thermoplastic elastomer with a high durability and flexibility.

### 6.3 Mechanical Properties

Resilience: Ability of a material to absorb energy when deformed elastically and to release the energy upon unloading.

Resilient: Will release substantially all of the energy when unloaded. Includes e.g. certain silicones, and thermoplastic elastomers.

Hardness: The ability of a material per se to resist deformation (e.g. described by a Young's Modulus, or an indentation hardness scale measured on a standardised sample size).
‘Soft’ materials may include silicone or thermo-plastic elastomer (TPE), and may, e.g. readily deform under finger pressure.
‘Hard’ materials may include polycarbonate, polypropylene, steel or aluminium, and may not e.g. readily deform under finger pressure.

Stiffness (or rigidity) of a structure or component: The ability of the structure or component to resist deformation in response to an applied load. The load may be a force or a moment, e.g. compression, tension, bending or torsion. The structure or component may offer different resistances in different directions.

Floppy structure or component: A structure or component that will change shape, e.g. bend, when caused to support its own weight, within a relatively short period of time such as 1 second.

Rigid structure or component: A structure or component that will not substantially change shape when subject to the loads typically encountered in use.
As an example, an I-beam may comprise a different bending stiffness (resistance to a bending load) in a first direction in comparison to a second, orthogonal direction. In another example, a structure or component may be floppy in a first direction and rigid in a second direction.

### 6.4 Anatomy

The following definitions correspond references identified in FIGS. 1-2.

### 6.4.1 Anatomy of the Face

Frankfort horizontal plane: A line extending from the most inferior point of the orbital margin to the left tragion. The tragion is the deepest point in the notch superior to the tragus of the auricle.

Sagittal plane: A vertical plane that passes from anterior (front) to posterior (rear). The midsagittal plane is a sagittal plane that divides the body into right and left halves.

### 6.4.2 Anatomy of the Skull

Frontal bone: The frontal bone includes a large vertical portion, the squama frontalis, corresponding to the region known as the forehead.

Occipital bone: The occipital bone is situated at the back and lower part of the cranium. It includes an oval aperture, the foramen magnum, through which the cranial cavity communicates with the vertebral canal. The curved plate behind the foramen magnum is the squama occipitalis.

Parietal bones: The parietal bones are the bones that, when joined together, form the roof and sides of the cranium.

Sphenoid bone: A wedge shaped bone of the base of the cranium.

Temporal bones: The temporal bones are situated on the bases and sides of the skull, and support that part of the face known as the temple.

Zygomatic bones: The face includes two zygomatic bones, located in the upper and lateral parts of the face and forming the prominence of the cheek.

### 6.5 User Interface

Frame: Frame will be taken to mean a display housing unit that bears the load of tension between two or more points of connection with a headgear and/or a hoop. The frame may seal against the user's face in order to limit and/or prevent the ingress and/or egress of light.

Hoop: Hoop will be taken to mean a form of positioning and stabilizing structure designed for use on a head. For example the hoop may comprise a collection of one or more struts, ties and stiffeners configured to locate and retain a user interface in position on a users' face for holding a display unit in an operational position in front of a user's face. Some ties are formed of a soft, flexible, elastic material such as a laminated composite of foam and fabric/textile. In some forms, the term headgear may be synonymous with the term hoop.

Membrane: Membrane will be taken to mean a typically thin element that has, preferably, substantially no resistance to bending, but has resistance to being stretched.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.

Stiffener: A stiffener will be taken to mean a structural component designed to increase the bending resistance of another component in at least one direction.

Strut: A strut will be taken to be a structural component designed to increase the compression resistance of another component in at least one direction.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees.

Tie (noun): A structure designed to resist tension.

### 6.6 Shape of Structures

Products in accordance with the present technology may comprise one or more three-dimensional mechanical structures, for example a mask cushion or an impeller. The three-dimensional structures may be bounded by two-dimensional surfaces. These surfaces may be distinguished using a label to describe an associated surface orientation, location, function, or some other characteristic. For example a structure may comprise one or more of an anterior surface, a posterior surface, an interior surface and an exterior surface. In another example, a seal-forming structure may comprise a face-contacting (e.g. outer) surface, and a separate non-face-contacting (e.g. underside or inner) surface. In another example, a structure may comprise a first surface and a second surface.

To facilitate describing the shape of the three-dimensional structures and the surfaces, we first consider a cross-section through a surface of the structure at a point, p. See FIG. 3A to FIG. 3E, which illustrate examples of cross-sections at point p on a surface, and the resulting plane curves. FIGS. 3A to 3E also illustrate an outward normal vector at p. The outward normal vector at p points away from the surface. In some examples we describe the surface from the point of view of an imaginary small person standing upright on the surface.

### 6.6.1 Curvature in One Dimension

The curvature of a plane curve at p may be described as having a sign (e.g. positive, negative) and a magnitude (e.g. 1/radius of a circle that just touches the curve at p).

Positive curvature: If the curve at p turns towards the outward normal, the curvature at that point will be taken to be positive (if the imaginary small person leaves the point p they must walk uphill). See FIG. 3A (relatively large positive curvature compared to FIG. 3B) and FIG. 3B (relatively small positive curvature compared to FIG. 3A). Such curves are often referred to as concave.

Zero curvature: If the curve at p is a straight line, the curvature will be taken to be zero (if the imaginary small person leaves the point p, they can walk on a level, neither up nor down). See FIG. 3C.

Negative curvature: If the curve at p turns away from the outward normal, the curvature in that direction at that point will be taken to be negative (if the imaginary small person leaves the point p they must walk downhill). See FIG. 3D (relatively small negative curvature compared to FIG. 3E) and FIG. 3E (relatively large negative curvature compared to FIG. 3F). Such curves are often referred to as convex.

### 6.6.2 Curvature of Two Dimensional Surfaces

A description of the shape at a given point on a two-dimensional surface in accordance with the present technology may include multiple normal cross-sections. The multiple cross-sections may cut the surface in a plane that includes the outward normal (a “normal plane”), and each cross-section may be taken in a different direction. Each cross-section results in a plane curve with a corresponding curvature. The different curvatures at that point may have the same sign, or a different sign. Each of the curvatures at that point has a magnitude, e.g. relatively small. The plane curves in FIGS. 3A to 3E could be examples of such multiple cross-sections at a particular point.

Principal curvatures and directions: The directions of the normal planes where the curvature of the curve takes its maximum and minimum values are called the principal directions. In the examples of FIG. 3A to FIG. 3E, the maximum curvature occurs in FIG. 3A, and the minimum occurs in FIG. 3E, hence FIG. 3A and FIG. 3E are cross sections in the principal directions. The principal curvatures at p are the curvatures in the principal directions.

Region of a surface: A connected set of points on a surface. The set of points in a region may have similar characteristics, e.g. curvatures or signs.

Saddle region: A region where at each point, the principal curvatures have opposite signs, that is, one is positive, and the other is negative (depending on the direction to which the imaginary person turns, they may walk uphill or downhill).

Dome region: A region where at each point the principal curvatures have the same sign, e.g. both positive (a “concave dome”) or both negative (a “convex dome”).

Cylindrical region: A region where one principal curvature is zero (or, for example, zero within manufacturing tolerances) and the other principal curvature is non-zero.

Planar region: A region of a surface where both of the principal curvatures are zero (or, for example, zero within manufacturing tolerances).

Edge of a surface: A boundary or limit of a surface or region.

Path: In certain forms of the present technology, ‘path’ will be taken to mean a path in the mathematical-topological sense, e.g. a continuous space curve from f(0) to f(1) on a surface. In certain forms of the present technology, a ‘path’ may be described as a route or course, including e.g. a set of points on a surface. (The path for the imaginary person is where they walk on the surface, and is analogous to a garden path).

Path length: In certain forms of the present technology, ‘path length’ will be taken to mean the distance along the surface from f(0) to f(1), that is, the distance along the path on the surface. There may be more than one path between two points on a surface and such paths may have different path lengths. (The path length for the imaginary person would be the distance they have to walk on the surface along the path).

Straight-line distance: The straight-line distance is the distance between two points on a surface, but without regard to the surface. On planar regions, there would be a path on the surface having the same path length as the straight-line distance between two points on the surface. On non-planar surfaces, there may be no paths having the same path length as the straight-line distance between two points. (For the imaginary person, the straight-line distance would correspond to the distance ‘as the crow flies’.)

### 6.6.3 Holes

A surface may have a one-dimensional hole, e.g. a hole bounded by a plane curve or by a space curve. Thin structures (e.g. a membrane) with a hole, may be described as having a one-dimensional hole. See for example the one dimensional hole in the surface of structure shown in FIG. 3F, bounded by a plane curve.

A structure may have a two-dimensional hole, e.g. a hole bounded by a surface. For example, an inflatable tyre has a two dimensional hole bounded by the interior surface of the tyre. In another example, a bladder with a cavity for air or gel could have a two-dimensional hole. In a yet another example, a conduit may comprise a one-dimension hole (e.g. at its entrance or at its exit), and a two-dimension hole bounded by the inside surface of the conduit. See also the two dimensional hole through the structure shown in FIG. 3H, bounded by a surface as shown.

### 6.7 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

1. A forehead support for a head-mounted display system, comprising:
a frame attachable to a head-mounted display unit of the head-mounted display system in use; and
a resilient forehead pad attached to the frame and configured to engage the user's forehead in use, and wherein at least a portion of the frame is able to flex to conform to the shape of the user's forehead in use; and
a shaping member attached to the frame, the shaping member configured to impart a shape to the frame.

2. The forehead support of claim 1, wherein the shaping member is more rigid than the frame.

3. The forehead support of claim 1, wherein the shaping member is substantially rigid.

4. The forehead support of claim 1, wherein the shaping member is configured to attach to an anterior surface of the frame.

5. The forehead support of claim 1, wherein the shaping member comprises a plate.

6. The forehead support of claim 1, wherein the shaping member comprises a skeleton.

7. The forehead support of claim 1, wherein the shape imparted to the frame by the shaping member corresponds to the shape of user's forehead.

8. The forehead support of claim 1, wherein the shape imparted to the frame by the shaping member matches the shape of the user's forehead.

9. The forehead support of claim 1, wherein the shaping member is formed based on a scan of the user's forehead.

10. The forehead support of claim 1, wherein the frame when shaped by the shaping member is configured to bias the forehead pad into a shape that is more curved than the user's forehead such that in use the forehead pad is conformed to the shape of the user's head.

11. The forehead support of claim 1, wherein the frame comprises a central portion structured to attach to the display unit housing, and

12. The forehead support of claim 11, wherein the frame comprises a pair of lateral portions having lateral ends able to move with respect to the central portion, the shaping member constructed and arranged to conform the lateral portions to the shape of the user's forehead in use.

13. The forehead support of claim 12, wherein the lateral ends are each able to be moved by the shaping member at least partially medially and laterally.

14. The forehead support of claim 12, wherein the lateral ends are each able to be moved by the shaping member at least partially anteriorly and posteriorly.

15. The forehead support of claim 12, wherein each of the lateral portions comprises a superior lateral portion and an inferior lateral portion and the lateral ends comprise a superior lateral end of each superior lateral portion and an inferior lateral end of each inferior lateral portion, each superior lateral portion being able to be flexed by the shaping member independently of the respective inferior lateral portion.

16. The forehead support of claim 11, wherein the frame comprises a superior medial portion having a superior end able to move with respect to the central portion of the frame, the shaping member constructed and arranged to conform the superior medial portion to the shape of the user's forehead in use.

17. The forehead support of claim 16, wherein the superior end is able to be moved by the shaping member at least partially anteriorly and posteriorly.

18. The forehead support of claim 11, wherein the frame comprises an inferior medial portion having an inferior end able to move with respect to the central portion of the frame, the shaping member constructed and arranged to conform the inferior medial portion to the shape of the user's forehead in use.

19. The forehead support of claim 18, wherein the inferior end is able to be moved by the shaping member at least partially anteriorly and posteriorly.

20. The forehead support of claim 1, wherein forehead pad comprises a foam layer.

21. The forehead support of claim 1, wherein forehead pad comprises a textile user-contacting layer.

22. A head-mounted display system, comprising:
a head-mounted display unit comprising a display unit housing comprising a display; and
a positioning and stabilising structure constructed and arranged to hold the head-mounted display unit in an operable position on the user's head in use;
wherein the positioning and stabilising structure comprises a forehead support, the forehead support comprising a frame attached to the head-mounted display unit in use and a resilient forehead pad attached to the frame and configured to engage the user's forehead in use, and wherein at least a portion of the frame is able to flex to conform to the shape of the user's forehead in use;
wherein the forehead support comprises a shaping member attached to the frame, the shaping member configured to impart a shape to the frame.
